(19) United States
(12) Patent Application Publication
(10) Pub. No.: US 2012/0237937 A1
(43) Pub. Date: Sep. 20, 2012

(54) METHODS TO DETECT CANCER IN ANIMALS

(75) Inventors: Teresa Whei-Mei Fan, Louisville, KY (US); Andrew Nicholas Lane, Louisville, KY (US); Michael Bousamra, Louisville, KY (US); Donald M. Miller, Louisville, KY (US); Richard M. Higashi, Prospect, KY (US)

(73) Assignee: UNIVERSITY OF LOUISVILLE RESEARCH FOUNDATION, INC., Louisville, KY (US)

(21) Appl. No.: 13/377,560

(22) PCT Filed: Jun. 11, 2010
(86) PCT No.: PCT/US10/38425

§ 371 Date: Jun. 4, 2012

Related U.S. Application Data
(60) Provisional application No. 61/186,572, filed on Jun. 12, 2009.

(51) Int. Cl. G01N 24/08; G01N 33/574; G01N 21/64; G01N 27/62

(52) U.S. Cl. 435/6.12; 435/34; 435/14; 435/7.4

(57) ABSTRACT

Some embodiments of the invention include methods for detecting the presence of cancer in animal tissue in an animal that was administered a labeled molecule. Some of the methods disclosed comprise obtaining an NMR spectrum, an MS spectrum, or both. In some instances, spectra can be taken of a cancer cell extract of the tissue and of a non-cancer cell extract of the tissue. In some embodiments, the amounts of at least one resultant labeled molecule (e.g., a molecule resulting from transformation of the administered labeled molecule) from each extract can be compared to detect the presence of cancer.

### CROSS-REFERENCE TO RELATED APPLICATIONS

This application claims the benefit of U.S. Provisional Application No. 61/186,572, filed Jun. 12, 2009, which is herein incorporated by reference in its entirety.

### GOVERNMENT RIGHTS

This invention was made in part with government support under the National Cancer Institute (grant numbers 1R01CA101199-01 and 1R01CA118434-01A2), the National Center for Research Resources (NIH Grant Number 5P20RR018733), and the National Science Foundation EPSCoR (grant numbers EPS-0132295 and EPS-0447479). The U.S. government has certain rights in the invention.

### BACKGROUND

Although measurements of gene expression and protein expression provide useful clues to metabolic dysfunctions in some cancers, they may not give a complete picture of metabolic changes that result in the cancer (e.g., malignant) phenotype. Posttranslational modifications, protein inhibitors, allosteric regulation by effector metabolites, alternative gene functions, or compartmentalization are some examples that result in metabolic changes that may not be reflected by gene expression or protein expression determination. Therefore, metabolic profiling (or metabolomic) investigations (which can be supplemented with protein expression or gene expression measurements) can be useful to study and detect cancer and cancer phenotypes.

### SUMMARY

Some embodiments of the invention include methods for detecting the presence of cancer in an animal that was administered an administered labeled molecule. In some instances the method comprises (i) obtaining a first NMR spectrum of a first non-cancer cell extract, and obtaining a second NMR spectrum of a first cancer cell extract, (ii) obtaining a first MS spectrum of a second non-cancer cell extract, and obtaining a second MS spectrum of a second cancer cell extract, or both. The first non-cancer cell extract was obtained from a first set of non-cancer cells removed from a tissue of the animal; the first cancer cell extract was obtained from a first set of cancer cells removed from the tissue of the animal; the second non-cancer cell extract was obtained from a second set of non-cancer cells removed from the tissue of the animal, and the second cancer cell extract was obtained from a second set of cancer cells removed from the tissue of the animal. A first amount of at least one resultant labeled molecule is determined from the first NMR spectrum, from the first MS spectrum, or from both. A second amount of at least one resultant labeled molecule is determined from the second NMR spectrum, from the second MS spectrum, or from both. Cancer can be detected by comparing the first amount of at least one resultant labeled molecule with the second amount of at least one resultant labeled molecule.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1. Panels A-D show the time course analysis of ¹³C-metabolites in plasma for patients #6-10. Panel E shows the ¹³C satellite pattern of the 3-methyl group of lactate (¹³C-CH₃-lac) in the 1-D ¹H NMR spectrum of patient #6 after 3 hr of [U-¹³C]-Glc infusion. The chemical shifts of lactate and Ala reflected the acidic pH of the trichloroacetic acid (TCA) extract. Panels F and G are comparisons of metabolite profiles in TCA extracts of paired normal and cancerous lung tissues of patient #6. The dashed lines trace metabolites that differed in abundance between cancerous and normal lung tissues.

FIG. 2. 2-D ¹H TOCSY identification of metabolites in the lung tumor tissue of patient #6 is displayed along with the corresponding 1-D high-resolution spectrum. Panels A,B and C,D show the 0.8-6.4 and 5.7-9.5 ppm regions of the spectra, respectively.

FIG. 3. Panel B is ¹H-¹³C 2-D HSQC identification of ¹³C-metabolites in the TCA extracts of lung tumor tissues of patient #6. Panel A is the 1-D projection spectrum of the 2-D data along the ¹³C dimension. Panel C displays the expanded spectral region of C3 and C4 resonances of Glu, Gln, and GSSG-Glu to illustrate the resolution of these resonances in the 2-D HSQC contour map.

FIG. 4. Comparison of metabolite profiles in TCA extracts of paired non-cancerous and cancerous lung tissues of patient #6. Metabolites in the 1-D ¹H NMR (panel A) and ¹³C HSQC projection spectra (panel B) were assigned as in FIGS. 2 and 3, respectively. The dashed lines trace metabolites that differed in abundance between cancerous and non-cancerous lung tissues.

FIG. 5. Relationships between Krebs cycle intermediates and glycolytic products in terms of ¹³C-labeled and total concentrations for lung tumor and non-cancerous tissues resected from patients #6-10, as determined by GC-MS analysis. The linear fit for cancerous tissue and non-cancerous tissue are represented by solid lines and dashed lines, respectively.

FIG. 6. Expected ¹³C labeling patterns in mitochondrial Krebs cycle intermediates and byproducts with [U-¹³C]-Glc as tracer. The cycle reactions are depicted without (panel A) or with (panel B) anaplerotic pyruvate carboxylase (PC) reaction. The ¹³C positional isotopomer patterns illustrated are the result of one cycle turn. The letter C's surrounded by circles represent ¹³C labeled carbons before scrambling. Squares and diamond shapes around the letter C's represent ¹³C labeled carbons after scrambling. The pyruvate with a rectangle around it denotes a separate pool of pyruvate. Solid and dashed arrows denote favorable single and multi-step reactions, respectively. Open arrows in panel A delineate ¹³C-labeled OAA after one turn from unlabeled pre-existing OAA.

FIG. 7. Western blotting (panel A) and image analysis (panel B) of PC protein patterns of paired tumor and non-cancerous tissues from patients #6-10. Normalized PC response represented PC image density normalized to α-tubulin image density. PC band for the rest of tissues was quantified using the same blot but with 17 min. of film exposure. N: non-cancerous; C: cancer; ND: not determined. The data shown is representative of two separate blot analyses. Panel C shows ratios of normalized Western Blot image analysis of tumor and non-cancerous tissue for patients #11-21 and #22b-28b. Three regions of patient 28b's lung tumor were analyzed. UL: Upper lobe lesion; LL: Lower lobe lesion.

FIG. 8. Glucose consumption (solid symbols) and lactate production (open symbols) in two SCID mice (circle symbols for one mouse and square symbols for the other mouse).

FIG. 9. GC-MS analysis of lactate isotopologues in the SCID mouse tissues of brain, heart, kidney, liver, lung, and muscle.

FIG. 10. Tracking of ¹³C atoms from ¹³C₆-glucose to ¹³C-lactate through glycolysis, pentose phosphate pathway, Krebs cycle, and gluconeogenesis. Panel A denotes the ¹³C flow through the non-oxidative branch of PPP and glycolysis in the presence of ¹³C₆-glucose+unlabeled glucose. Panel B tracks ¹³C atoms from glycolysis, Krebs cycle, gluconeogenesis, and glycolysis again. Black dots represent unlabeled carbons while dots with shapes around them denote ¹³C. Dots surrounded by square and diamond shapes in panel B illustrate scrambled ¹³C. Dashed black arrows denote multiple reactions steps, double-headed black arrows indicate reversible reactions, and dashed black arrows crossing molecule bonds denote site of carbon-carbon bond breaking PDH: pyruvate dehydrogenase; SCS: succinyl CoA synthetase; PEPCK: phosphoenolpyruvate carboxykinase; OAA: oxaloaceate; α-KG: α-ketoglutarate; PEP: phosphoenolpyruvate.

FIG. 11. GC-MS analysis of Ala isotopologues in the SCID mouse tissues of brain, heart, kidney, liver, lung, and muscle.

FIG. 12. GC-MS analysis of succinate isotopologues in the SCID mouse tissues of brain, heart, kidney, liver, lung, and muscle.

FIG. 13. GC-MS analysis of Asp isotopologues in the SCID mouse tissues of brain, heart, kidney, liver, lung, and muscle.

FIG. 14. GC-MS analysis of Glu isotopologues in the SCID mouse tissues of brain, heart, kidney, liver, lung, and muscle.

FIG. 15. GC-MS analysis of Gln isotopologues in the SCID mouse tissues of brain, heart, kidney, liver, lung, and muscle.

FIG. 16. 2-D ¹H-¹³C HSQC-TOCSY analysis of SCID mouse lung extracts. Rectangular boxes traced some of the covalent linkages represented by the 2-D cross-peaks. GSH: reduced glutathione; Glc: glucose; PCr-NMe, PC-NMe: N-methyl carbon of phosphocreatine or phosphocholine, respectively; AXP: adenine nucleotides

FIG. 17. 1-D HSQC spectral comparison of six SCID mouse tissue extracts after mouse infusion with labeled ¹³C₆-glucose. The 1-D HSQC spectra of all six tissues were normalized to tissue residue weight (remained after polar and lipid extractions) and spectral parameters so that the intensity of metabolite resonances reflected their tissue content.

FIG. 18. 1-D HSQC spectral comparison of SCID mouse tumorous lung tissue and SCID mouse normal lung tissue. The 1-D HSQC spectra were normalized to tissue residue weight (remained after polar and lipid extractions) and spectral parameters so that the intensity of metabolite resonances reflected their tissue content.

FIG. 19. GC-MS analysis of ¹³C— isotopologue series of metabolites in tumorous and normal lung tissues of SCID mice. The values displayed are the difference between tumorous lung tissue extracts and normal lung tissue extracts in μmole/g dry residue weight. The dry residue weight was obtained from tissue remained after polar and lipid extractions, described above. Symbols +1, +2, +3, +4, and +5 are differences for the singly, doubly, triply, quadruply, and quintuply ¹³C-labeled isotopologues, respectively. T is the difference in total metabolite amount (i.e., labeled and unlabeled metabolite).

### DETAILED DESCRIPTION

Some embodiments of the invention include methods for detecting the presence of cancer in an animal that was administered an administered labeled molecule. In some instances the method comprises (i) obtaining a first NMR spectrum of a first non-cancer cell extract, and obtaining a second NMR spectrum of a first cancer cell extract, (ii) obtaining a first MS spectrum of a second non-cancer cell extract, and obtaining a second MS spectrum of a second cancer cell extract, or both. The first non-cancer cell extract was obtained from a first set of non-cancer cells removed from a tissue of the animal; the first cancer cell extract was obtained from a first set of cancer cells removed from the tissue of the animal; the second non-cancer cell extract was obtained from a second set of non-cancer cells removed from the tissue of the animal, and the second cancer cell extract was obtained from a second set of cancer cells removed from the tissue of the animal. A first amount of at least one resultant labeled molecule is determined from the first NMR spectrum, from the first MS spectrum, or from both. A second amount of at least one resultant labeled molecule is determined from the second NMR spectrum, from the second MS spectrum, or from both. Cancer can be detected by comparing the first amount of at least one resultant labeled molecule with the second amount of at least one resultant labeled molecule.

In some embodiments of the invention, labeled molecules and isotopomer approaches can be used to study animals with cancer. For example, metabolic differences can be investigated by infusing labeled molecules (e.g., labeled metabolites) into animals with cancer, followed by removal and processing of paired non-cancerous cells and cancerous cells of the animal's tissue. NMR, MS, or both can be used for isotopomer-based metabolomic analysis of the extracts of tissues. Labeled molecules can be, for example, administered intravenously into an animal prior to removal (e.g., surgical resection) of the cancer (e.g., primary tumor) cells and non-cancerous cells in the tissue. In some embodiments, the non-cancerous cells are taken from cells surrounding the cancerous cells in the tissue. In some instances, differences in metabolic transformations between the non-cancerous cells and cancer cells can be determined using NMR, MS, or both. In some embodiments, this approach can be used to detect cancer (e.g., in some instances including analysis of metabolic traits of cancer cells) without interferences from either intrinsic (e.g. genetic) or external environmental factors (e.g. diet) because the patient's own non-cancerous cells serve as an internal control.

As used herein, the term “metabolite” refers to the reactants (e.g., precursors), intermediates, and products of metabolic transformations.

Some embodiments of the invention include methods for detecting the presence of cancer in an animal that was administered an administered labeled molecule.

The route of administration of the administered labeled compound may be of any suitable route including, but are not limited to an oral route, a parenteral route, a cutaneous route, a nasal route, a rectal route, a vaginal route, or an ocular route. The choice of administration route can depend on the compound identity, such as the physical and chemical properties of the compound, as well as the age and weight of the animal, the particular cancer, degree of localization or encapsulation of the cancer, or the severity of the cancer. Of course, combinations of administration routes can be administered, as desired.

The administered labeled molecule can be any suitable labeled molecule, including but not limited to a ¹³C isotopomer of glucose, a ¹³C isotopomer of pyruvate, a ¹³C isotopomer of Ala, an ¹⁵N isotopomer of Ala, a ¹³C isotopomer of acetate, a ¹³C isotopomer of glutamine, an ¹⁵N isotopomer of glutamine, glucose (C1, C2, C3, C4, C5, C6, or any combination thereof that are ¹³C labeled; e.g., all glucose carbons are ¹³C labeled, ¹³C₁-glucose, ¹³C₂-glucose, ¹³C₃-glucose, ¹³C₄-glucose, or ¹³C₅-glucose), pyruvate (C1, C2, C3 or any combination thereof are ¹³C labeled; e.g., C1, C2, and C3 are all ¹³C labeled, ¹³C₁-pyruvate, or ¹³C₂-pyruvate), acetyl CoA (C1, C2 or both are ¹³C labeled; e.g., ¹³C₁-acetyl CoA), and Ala (C1, C2, C3 or any combination thereof are ¹³C labeled; e.g., C1, C2, and C3 are all ¹³C labeled, ¹³C₁-Ala, or ¹³C₂-Ala), ¹³C labeled glycerol, ¹³C labeled fatty acids (e.g., octanoic acid), ¹³C labeled amino acids (e.g., glutamine, serine, tryptophan that have one or more labels), or ¹⁵N labeled amino acids (e.g., glutamine, serine, tryptophan that have one or more labels). The administered labeled molecule can also include molecules (e.g., amino acids) that have one or more ¹⁵N labels, including but not limited to, ¹⁵N labeled amino acids. Of course, the administered labeled molecule can be labeled with one or more ¹³C labels, one or more ¹⁵N labels, one or more ²H, one or more ³H, one or more ⁷⁷Se, one or more ³¹P, or combinations thereof. The designation “¹³Cₓ” indicates that x of the molecule's carbons are ¹³C labeled, but when x is less than the total number of the molecule's carbons the specific labeling locations are not designated and ¹³Cₓ refers to a set of molecules. For example, ¹³C₅-glucose is the set of five glucose molecules that have 5 of the 6 carbons ¹³C labeled. When all carbons in a molecule are ¹³C-labeled that is designated as being uniformly labeled and is indicated by [U-¹³C]. In some embodiments, the administered labeled molecule is of uniformly ¹³C-labeled glucose ([U-¹³C]-Glc), ¹³C₁-glucose, ¹³C₂-glucose, ¹³C₃-glucose, ¹³C₄-glucose, ¹³C₅-glucose, [U-¹³C]-pyruvate, ¹³C₁-pyruvate, ¹³C₂-pyruvate, [U-¹³C]-acetyl CoA, ¹³C₁-acetyl CoA, [U-¹³C]-Ala, ¹³C₁-Ala, or ¹³C₂-Ala.

The amount of administered labeled molecule administered to the animal can be any suitable amount, including but not limited to about 10 mmol, about 25 mmol, about 50 mmol, about 75 mmol, about 100 mmol, about 125 mmol, about 150 mmol, about 175 mmol, about 200 mmol, about 400 mmol, about 600 mmol, about 700 mmol, or about 1000 mmol. The administered labeled molecule can be administered to the animal in a bolus administration or over a period of time. The amount of time the administered labeled molecule can be administered to the animal can be any suitable time, including but not limited to about 1 min., about 3 min., about 5 min., about 10 min., about 20 min., about 30 min., about 40 min., about 50 min., about 1 hour, about 2 hours, or about 5 hours. The amount and time of administration can, in some embodiments, depend upon one or more of the administered label molecule, the resultant labeled molecule, the animal, the tissue, the cancer to be detected, the health of the animal, the age and weight of the animal, the enzyme or metabolic pathway to be analyzed, or any other relevant factor.

Animals include but are not limited to primates, canine, equine, bovine, porcine, ovine, avian, or mammalian. In some embodiments, the animal is a human, dog, cat, horse, cow, pig, sheep, chicken, turkey, mouse, or rat.

The cancer (including cancerous tumors) can include but is not limited to carcinomas, sarcomas, hematologic cancers, neurological malignancies, basal cell carcinoma, thyroid cancer, neuroblastoma, ovarian cancer, melanoma, renal cell carcinoma, hepatocellular carcinoma, breast cancer, colon cancer, lung cancer, pancreatic cancer, brain cancer, prostate cancer, chronic lymphocytic leukemia, acute lymphoblastic leukemia, rhabdomyosarcoma, Glioblastoma multiforme, meningioma, bladder cancer, gastric cancer, Glioma, oral cancer, nasopharyngeal carcinoma, kidney cancer, rectal cancer, lymph node cancer, bone marrow cancer, stomach cancer, uterine cancer, leukemia, basal cell carcinoma, cancers related to epithelial cells, or cancers that can alter the regulation or activity of PC. Cancerous tumors include, for example, tumors associated with any of the above mentioned cancers.

In some embodiments, cancerous tissue cells and non-cancerous tissue cells are removed from the animal. In some embodiments, the non-cancerous cells are taken from cells that are nearby or surrounding the cancerous cells in the tissue. In still other embodiments, the non-cancerous cells are taken from the same tissue from a different part of the animal (e.g., from a contralateral lung or breast). The removed cancerous tissue cells and the removed non-cancerous cells can be each extracted using the same or different extraction methods or solutions. The amount of at least one resultant molecule (e.g., a molecule resulting from the animal's body transforming the administered labeled molecule) is determined in the cancer cell extract and the non-cancer cell extract using NMR, MS or both. The presence of cancer can be determined by comparing the amount of at least one resultant molecule in the cancer cell extract with the amount of at least one resultant molecule in the non-cancer cell extract. Of course, extraction methods and solutions used for preparing NMR samples may or may not be different from extraction methods and solutions used for preparing MS samples.

The tissue can be any animal tissue including (e.g., mammalian tissues), such as but not limited to connective tissue, muscle tissue, nervous tissue, adipose tissue, endothelial tissue, or epithelial tissue. The tissue can be at least part of an organ or part of an organ system. Organs can include, but are not limited to heart, blood, blood vessels, salivary glands, esophagus, stomach, liver, gallbladder, pancreas, large intestines, small intestines, rectum, anus, colon, endocrine glands (e.g., hypothalamus, pituitary, pineal body, thyroid, parathyroids and adrenals), kidneys, ureters, bladder, urethraskin, hair, nails, lymph, lymph nodes, lymph vessels, leukocytes, tonsils, adenoids, thymus, spleen, muscles, brain, spinal cord, peripheral nerves, nerves, sex organs (e.g., ovaries, fallopian tubes, uterus, vagina, mammary glands, testes, vas deferens, seminal vesicles, prostate and penis), pharynx, larynx, trachea, bronchi, lungs diaphragm, bones, cartilage, ligaments, or tendons. Organ systems can include, but are not limited to circulatory system, digestive system, endocrine system, excretory system, integumentary system, lymphatic system, muscular system, nervous system, reproductive system, respiratory system, or skeletal system.

The tissue has cells that are cancerous and cells that are non-cancerous. Tissue cells can be removed from the animal by any suitable methods, including but not limited to surgical methods (e.g., resection), biopsy methods, or animal sacrifice followed by organ removal and dissection. The non-cancerous tissue cells are removed from any suitable distance from the cancerous portion of the tissue (e.g., the tumor margin) and can be, but is not limited to at least about 1 mm, at least about 3 mm, at least about 5 mm, at least about 1 cm, at least about 2 cm, or at least about 3 cm from the cancerous portion of the tissue. In some instances, the non-cancerous tissue cells are taken from the same tissue from a different part of the animal (e.g., from a contralateral lung or breast). In some embodiments, the non-cancerous tissue cells are completely free from or substantially (e.g., 99.9%, 99%, 95%, or 90%) free from cancerous cells. Removed tissue cells can be frozen in liquid nitrogen. Preparation of the removed tissue cells can be performed in any suitable manner (e.g., including pulverizing or grinding the tissue) to obtain the resultant labeled molecule and can include one or more extractions with solutions comprising any suitable solvent or combinations of solvents, such as, but not limited to acetonitrile, water, chloroform, methanol, butylated hydroxytoluene, trichloroacetic acid, or combinations thereof.

In some embodiments, tissue cells are removed at a certain time after administration of the administered labeled molecule. The time between the last administration of the administered labeled molecule and the removal of the tissue cells can be any suitable time, including but not limited to about 1 min., about 5 min., about 10 min., about 15 min., about 20 min., about 30 min., about 40 min., about 50 min., about 1 hour, about 2 hours, about 3 hours, about 4 hours, about 6 hours, about 9 hours, about 12 hours, or about 20 hours. For example, the time can be at least about 1 min., at least about 5 min., at least about 10 min., no more than about 20 min., no more than about 30 min., no more than about 40 min., no more than about 1 hour, no more than about 5 hours, or no more than about 20 hours. The amount of time can, in some embodiments, depend upon one or more of the administered labeled molecule, the resultant labeled molecule, the animal, the tissue, the cancer to be detected, the health of the animal, the enzyme or metabolic pathway, or any other relevant factor.

The resultant labeled molecule can result from a transformation of the administered labeled molecule. In some embodiments, this transformation occurs by enzymatic action or by action via a metabolic pathway or an anaplerotic pathway. Such metabolic pathways can include, but are not limited to Krebs cycle (also known as the citric acid cycle), glycolysis, pentose phosphate pathway (oxidative and non-oxidative) (PPP), gluconeogenesis, lipid biosynthesis, amino acid syntheses (e.g., synthesis of non-essential amino acids), catabolic pathways, urea cycle, Cori cycle, or glutamate/glutamine cycle. Enzymes involved in the transformation can include, but are not limited to pyruvate carboxylase (PC), succinyl CoA synthetase (SCS), phosphoenolpyruvate carboxykinase (PEPCK), transketolase, transaldolase, pyruvate dehydrogenase (PDH), a dehydrogenase (DH), glutaminase (GLS), isocitrate dehydrogenase (IDH), α-ketoglutarate dehydrogenase (OGDH), mitochondrial malate dehydrogenase (MDH), succinate dehydrogenase (SDH), fumarate hydratase (FH), hexokinase II (HKII), glyceraldehyde-3-phosphate dehydrogenase (GAPDH), phosphoglycerate kinase 1 (PGK-1), lactate dehydrogenase 5 (LDH-5), phosphofructokinase 1 and 2 (PFK-1 and PFK-2), glutathione peroxidase (GPx), or glutathione-5-transferase (GST). In some embodiments, the resultant labeled molecule is an isotopomer of any of lactate, alanine (Ala), arginine (Arg), serine (Ser), proline (Pro), asparagine (Asn), Glycine (Gly), glutamate (Glu), oxidized glutathione (GSSG), Glu-GSSH, Glu-GSH, glutamine (Gln), γ-aminobutyrate (GAB), succinate, citrate, isocitrate, fumarate, malate, aspartate (Asp), creatine (Cr), oxaloacetate: (OAA), α-ketoglutarate: (αKG), phosphocholine (P-choline), N-methyl-phosphocholine, taurine, glycogen, phenylalanine (Phe), tyrosine (Tyr), myo-inositol, α- and β-glucose, NAD⁺, cytosine nucleotides (CXP), uracil nucleotides (UXP), guanine nucleotides (GXP), or adenine nucleotides (AXP). In other embodiments, the resultant labeled molecule is uniformly ¹³C labeled lactate ([U-¹³C]-lactate), [U-¹³C]-Ala, ¹³C-3-Glu, ¹³C-3-Gln, ¹³C-3-glutamyl residue of oxidized glutathione (Glu-GSSG), ¹³C-2-Glu-GSSG, ¹³C-2-Glu, ¹³C-2-Asp, of ¹³C-3-Ala, ¹³C-3-lactate, ¹³C-3-Glu, ¹³C-3-Gln, ¹³C-3-Asp, ¹³C-2,3-succinate, ¹³C-2,4-citrate, ¹³C-1′-ribose-5′AXP, ¹³C-1-α- and -β-glucose, ¹³C-2,3-lactate, ¹³C-2 to 4-Glu, ¹³C-4-Gln, ¹³C-4-GSSG, ¹³C-4-GSG, ¹³C-2,4-citrate, ¹³C-2,3-Asp, ¹³C-1′,4′,5′-5′-AXP, ¹³C-1′,4′-5′-UXP, ¹³C-2-γ-aminobutyrate, ¹³C-3-γ-aminobutyrate, ¹³C-4-γ-aminobutyrate, ¹³C-3-Pro, or ¹³C-4-Pro.

Measurements using mass spectrometry system can result in the detection of collections of isotopomers with the same molecular mass, termed isotopologues. In some embodiments, at least one resultant labeled molecule will represent collections of isotopologues. Isotopologues can include but are not limited to any collection of isotopomers resulting from the transformation of an administered labeled molecule. For example, at least one resultant labeled molecule can be, but is not limited to ¹³C₂-lactate, ¹³C₃-lactate, ¹³C₂-Ala, ¹³C₃-Ala, ¹³C₂-succinate, ¹³C₃-succinate, ¹³C₄-succinate, ¹³C₂-Asp, ¹³C₃-Asp, ¹³C₄-Asp, ¹³C₂-Glu, ¹³C₃-Glu, ¹³C₄-Glu, ¹³C₅-Glu, ¹³C₂-Gln, ¹³C₃-Gln, ¹³C₄-Gln, ¹³C₅-Gln, ¹³C₂-fumarate, ¹³C₃-fumarate, ¹³C₄-fumarate, ¹³C₂-malate, ¹³C₃-malate, ¹³C₄-malate, ¹³C₂-Pro, ¹³C₃-Pro, ¹³C₄-Pro, ¹³C₅-Pro, ¹³C₂-Gly, ¹³C₃-Gly, ¹³C₂-Ser, ¹³C₃-Ser, ¹³C₂-pyruvate, ¹³C₃-pyruvate, ¹³C₂-citrate, ¹³C₃-citrate, ¹³C₂-isocitrate, or ¹³C₃-isocitrate.

The amount of one or more molecules of at least one resultant molecule can be any amount detectable including but not limited to about 0.001 μmol/g dry tissue weight, 0.01 μmol/g dry tissue weight, 0.1 μmol/g dry tissue weight, about 1 μmol/g dry tissue weight, about 2 μmol/g dry tissue weight, about 5 μmol/g dry tissue weight, about 10 μmol/g dry tissue weight, about 50, μmol/g dry tissue weight, about 100 μmol/g dry tissue weight, about 200 μmol/g dry tissue weight, about 300 μmol/g dry tissue weight, or about 500 μmol/g dry tissue weight. The amount of one or more of at least one resultant molecule can, in some embodiments, depend upon one or more of the administered label molecule, the resultant labeled molecule, the animal, the tissue, the cancer to be detected, the health of the animal, the enzyme or metabolic pathway, or any other relevant factor.

Using NMR, the spectrum of a cancer cell extract and a spectrum of an extract of a non-cancer cell extract are obtained. The type of NMR spectrum can be any suitable spectrum type to determine the amount of at least one resultant molecule, including but not limited to, 1-D ¹H, 1-D ¹³C, 1-D ¹⁵N, total correlation spectroscopy (TOCSY) (e.g., 2-D ¹H TOCSY), COSY (and any COSY variants), NOESY, EXSY, or heteronuclear correlation scalar coupling experiments, such as, but not limited to, heteronuclear single quantum coherence spectroscopy (HSQC) (e.g., ¹H-¹³C₂-D HSQC, ¹H1-D HSQC), SE-HSQC, CT-HSQC, HSQC-TOCSY (e.g., ¹H-¹³C₂-D HSQC-TOCSY), TROSY, HETCOR, COLOC, SECSY, FOCSY, J-resolved, INADEQUATE, HMQC, HMBC, HCACO, HCC, or CC-TOCSY. NMR spectra can include those collected, for example, using 1-D, 2-D, 3-D, or 4-D NMR techniques. NMR spectra can include those that are based on scalar coupling, dipolar coupling, or both. NMR spectra can also include spectra obtained using solid state techniques, including but not limited to those using magic angle spinning.

Using a mass spectrometry system, the MS spectrum of a cancer cell extract and a spectrum of a non-cancer cell extract are obtained. The mass spectrometry system can comprise the usual components of a mass spectrometer (e.g., ionization source, ion detector, mass analyzer, vacuum chamber, and pumping system) and other components, including but not limited to interface chromatography systems. The mass spectrometer can be any suitable mass spectrometer for determining the at least one resultant molecule. The mass analyzer system can include any suitable system including but not limited to, time of flight analyzer, quadrupole analyzer, magnetic sector, Orbitrap, linear ion trap, or fourier transform ion cyclotron resonance (FTICR). The ionization source can include, but is not limited to electron impact (EI), electrospray ionization (ESI), chemical ionization (CI), collisional ionization, natural ionization, thermal ionization, fast atom bombardment, inductively coupled plasma (ICP), or matrix-assisted laser desorption/ionization (MALDI). Interfaced chromatography systems can include any suitable chromatography system, including but not limited to gas chromatography (GC), liquid chromatography (LC), or ion mobility (which can be combined with LC or GC methods). In some instances, direct infusion can be used. In some instances the mass spectrometry system is GC/MS or LC/MS.

Once the NMR spectra, MS spectra, or both are obtained, the spectra are analyzed to determine the amount (e.g., the presence) of at least one resultant labeled molecule in the cancer cell extract and to determine the amount (e.g., the presence) of at least one resultant labeled molecule in the non-cancer cell extract.

For NMR spectra, analysis can include any suitable analysis to determine the amount of one or more resultant labeled molecule (such as, the number and position of labels in the resultant labeled molecule) including the determination of one or more NMR spectral characteristics, which include but are not limited to chemical shift, coupling patterns (e.g., dipolar coupling or spin-spin coupling, such as J-coupling), covalent linkage patterns, peak intensities, peak integrations (e.g., in a 1-D spectrum, in a projection spectrum of a 2-D spectrum, or cross peak integration in a 2-D spectrum) and the presence, extent, and quantification (e.g., peak intensity or peak integration) of satellite peaks (e.g., as a result of the splitting of ¹H spectra by ¹³C). In some instances, the analysis can include a comparison of one or more NMR spectral characteristics with that of a database (e.g., a database of standards).

For MS spectra, analysis can include any suitable analysis to determine the amount of one or more resultant labeled molecule (such as, the number and position of labels in the resultant labeled molecule) including analysis of one or more characteristics, which include but are not limited to chromatographic retention times (e.g., for GC/MS or LC/MS), and mass fragmentation patterns. In some instances, the analysis can include a comparison of characteristics with that of a database (e.g., a database of standards).

The method can further comprise the determination of the protein expression, gene expression, or both of proteins or their genes. Any suitable protein (or its gene) expression can be determined, including but not limited to PC, SCS, PEPCK, transketolase, transadolase, PDH, DH, GLS, IDH, OGDH, MDH, SDH, FH, HKII, GAPDH, PGK-1, LDH-5, PFK-2, GST, or proteins associated with metabolic pathways such as, but are not limited to Krebs cycle (also known as the citric acid cycle), glycolysis, pentose phosphate pathway (oxidative and non-oxidative), gluconeogenesis, lipid biosynthesis, amino acid syntheses (e.g., synthesis of non-essential amino acids), catabolic pathways, urea cycle, Cori cycle or glutamate/glutamine cycle. Protein expression can be determined by any suitable technique including, but not limited to techniques comprising gel electrophoresis techniques (e.g., Western blotting), chromatographic techniques, antibody-based techniques, centrifugation techniques, or combinations thereof. Gene expression can be determined by any suitable technique including, but not limited to techniques comprising PCR based techniques (e.g., real-time PCR), gel electrophoresis techniques, chromatographic techniques, antibody-based techniques, centrifugation techniques, or combinations thereof. Methods for measuring gene expression can comprise measuring amounts of cDNA made from tissue-isolated RNA.

In some embodiments of the invention, some metabolites can be found at higher levels in cancer cells than their surrounding non-cancerous cells. In other embodiments, some metabolites can be found at lower levels in cancer cells than their surrounding non-cancerous cells. For example, a ¹³C-enrichment in lactate, Ala, succinate, Glu, Asp, and citrate that is higher in the cancer cells can suggest more active glycolysis and Krebs cycle in the cancer cells. In other examples, enhanced production of the Asp isotopomer with three ¹³C-labeled carbons and the buildup of ¹³C-2,3-Glu isotopomer in cancer tissues can be observed. This enhanced production can be consistent with the transformations of glucose into Asp or Glu via glycolysis, anaplerotic pyruvate carboxylation (PC), and the Krebs cycle.

In still other embodiments, PC activation in cancer tissues can be found. Without wishing to be bound by theory, such PC activation may assist in replenishing the Krebs cycle intermediates which can be diverted to lipid, protein, and nucleic acid biosynthesis to fulfill the high anabolic demands for growth in lung tumor tissues. The metabolites, if so produced from such diversions, may be detected using the methods of the present invention.

### EXAMPLES

A. Human Studies

Patient Treatment and Sample Collection

Lung cancer patients were recruited based on the criteria of surgical eligibility and no history of diabetes. Each patient was consented in accordance with the U.S. HIPAA regulations. The following patient protocol was approved by the Internal Review Board at the University of Louisville. Ten grams uniformly ¹³C labeled glucose ([U-¹³C]-Glc or ¹³C₆-Glc or ¹³C₆-Glucose) in sterile saline solution were infused i.v. over a 30 min time period into each patient in the pre-op room approximately 3 or 12 hr prior to resection. Whole blood samples were collected into a vacutainer containing the anticoagulant K₃EDTA or K₃-EDTA before and after [U-¹³C]-Glc infusion as well as after the surgery. Additional blood samples were collected 3 and 12 hr after the [U-¹³C]-Glc infusion for the 12 hr treatment cases. To minimize metabolic changes, all tissue and blood samples were collected at the operating table with a comparable delay before liquid N₂ freezing. The blood samples were placed on ice immediately after collection and centrifuged at 4° C. at 3,500×g for 15 min to recover the plasma fraction. All blood samples were aliquoted and flash-frozen in liquid N₂ within 30 min of collection. Potassium EDTA was used as an anti-coagulant to minimize metabolic artifacts associated with anti-coagulation; it also served to remove the influence of interfering cations such as paramagnetic Fe³⁺ and Cu²⁺ for NMR analysis.

All timings from first incision to cutting arteries and veins to the lung were recorded so that the period of ischemia could be determined immediately after tissue resection, excess blood was blotted from the tissue, and small pieces of non-cancerous and tumor tissue were cut by the surgeon and freeze-clamped in liquid N₂ within 5 minutes of removal from the chest cavity. The freezing process arrested metabolic changes almost instantaneously. In all cases, the tumors were well differentiated so that the extent of the tumor was assessed visually and by palpation. Non-cancerous tissue was removed at least 2 cm from the tumor margin and certified to be tumor-free by trained pathologists. Subsequent pathological evaluation also confirmed tumor status and provided the tumor stage. All samples were stored at −80° C. until further processing for analysis.

Tissue Processing and Extraction

Frozen tissue samples were pulverized into <10 μm particles in liquid N₂ using a Spex freezer mill (Spex CertiPrep, Inc., Metuchen, N.J.) to maximize efficiency for subsequent extraction while maintaining biochemical integrity. An aliquot of the frozen powder was lyophilized before extraction for metabolites.

Water-soluble or polar metabolites were extracted from lyophilized tissue powders (4-48 mg) in ice-cold 10% trichloroacetic acid (TCA) (v/w minimum 40/1) while leaving proteins, nucleic acids, and polysaccharides behind, as described previously (Kim et al. Antioxidants & Redox Signaling 2001, 3:361-373). The extraction was performed twice for quantitative recovery. An aliquot (150 μl) of plasma samples were made to final 10% TCA concentration to precipitate proteins and recover metabolites in the supernatant. TCA was then removed from tissue or plasma extracts by lyophilization. The dry pellet was dissolved in nanopure water and two small aliquots were lyophilized for silylation and GC-MS analysis while the remaining bulk was passed through a Chelex 100 resin column (Bio-Rad Laboratories, Inc., Hercules, Calif.) to neutralize and remove interfering multivalent cations for NMR analysis.

NMR Analysis

The ¹H reference standard, DSS (2,2-Dimethyl-2-silapentane-5-sulfonate sodium salt) was added (30 or 50 nmoles) to the TCA extracts of tissue or plasma samples, respectively. NMR analysis of the TCA extracts was performed at 20° C. on a Varian Inova 14.1 T NMR spectrometer (Varian, Inc., Palo Alto, Calif.) equipped with a 5-mm HCN triple resonance cold probe. The following NMR experiments were conducted for the determination of metabolite and ¹³C positional isotopomers: 1-D ¹H, 2-D ¹H TOCSY, 2-D ¹H-¹³C HSQC and HSQC-TOCSY. ¹H and ¹³C chemical shifts of the TCA extracts were referenced to DSS at 0.00 ppm and indirectly to the ¹H shift, respectively. Various metabolites were identified based on their ¹H and ¹³C chemical shifts, ¹H coupling patterns, as well as ¹H-¹H and ¹H-¹³C covalent linkage patterns (acquired from the TOCSY and HSQC experiments, respectively), in comparison with those in our in-house standard database. See Fan et al. (2008) Prog. NMR Spectrosc. 52, 69-117; Fan (1996) Prog. NMR Spectrosc 28, 161-219.

For metabolite and ¹³C-isotopomer quantification, selected ¹H peaks in the 1-D NMR spectra were deconvoluted and integrated using MacNuts software (Acorn NMR, Inc., Livermore, Calif.). The resulting intensity of peaks of interest was calibrated by the peak intensity of DSS for absolute quantification. Percentage ¹³C abundance of labeled metabolites at specific carbon positions was quantified by integrating appropriate 13C satellite peaks in 1-D 1H or 2-D TOCSY spectra, as previously described (Lane et al. Metabolomics 2007, 3:79-86; Fan et al. Progress in NMR Spectroscopy 2008, 52:69-117.).

GC-MS Analysis

The same extracts from NMR analysis were subjected to GC-MS analysis for quantifying total and ¹³C-labeled mass isotopomers, as described in full previously (Fan et al. Metabolomics Journal 2005, 1:325-339). Briefly, the lyophilized extract was derivatized in MTBSTFA (N-methyl-N-[tert-butyldimethylsilyl]trifluoroacetamide) (Regis Chemical, Morton Grove, Ill.) and the tert-butyldimethylslylyl derivatives were separated and quantified on a PolarisQ GC-ion trap MSn (ThermoFinnigan, Austin, Tex.) equipped with a 50 m×0.15 mm i.d. open tubular column with 0.4 μm coat BPX-5 (5% phenyl/methyl equivalent) (SGE, Austin, Tex.). Metabolites were identified based on their GC retention times and mass fragmentation patterns by comparison with those of the standards. Absolute quantification of metabolites was done by calibrating the response of selected ions characteristic of a given metabolite from sample runs with that from standard runs (Fan et al. Metabolomics Journal 2005, 1:325-339). Relative metabolite abundances were calculated using Xcalibur (ThermoFinnigan, San Jose, Calif.) or Met-IDEA software to extract peak areas of individual ions characteristic of each component. For Met-IDEA, default program settings for ion trap mass spectrometer were used in the data analysis except for a mass accuracy m/z set at 0.001 and a mass range set on either side of the target m/z at ±0.6. For Xcalibur, peak detection, identification, background subtraction, and quantification was performed using parameters custom-tuned to each analyte. Quantification of mass isotopomers was conducted by subtraction of the isotopic profile at natural-abundance (determined empirically from the analyses of standards) from that of the sample. This procedure was repeated for each series of mass isotopomers to arrive at the ¹³C-enriched profiles (Fan et al. Metabolomics Journal 2005, 1:325-339). In all cases, the pseudo-molecular ion cluster, characteristic of MTBSTFA-derivatives, was used to ensure that true mass isotopomers were quantified.

Gene Expression Analysis

From an initial gene microarray analysis of a separate set of six paired tissue samples, a number of statistically significant gene expression differences were discerned between lung cancer tissues and their non-cancerous counterparts. This included an over expression of the pyruvate carboxylase (PC) gene in lung cancer versus paired non-cancerous tissues. Due to sample limitation, the array analysis was not performed for patients #6-10.

Real time (RT)-PCR was used instead to probe PC gene expression in patients #6-10. Non-cancerous and cancer tissue RNA was isolated using RNeasy minikit (Qiagen) primarily following the manufacturer's instruction. The only modification was that Trizol Reagent (invitrogen), instead of Buffer RLT provided by the kit, was used to disrupt and homogenize the pulverized frozen tissues at the first step of RNA isolation. The modified procedure was found to provide a higher RNA yield and better purity. The integrity of RNA was confirmed by 1% agarose gel electrophoresis. RNA was reverse transcribed into first strand cDNA using oligo(dT)₁₈ and SuperScript II reverse transcriptase (Invitrogen). Specifically, 1 μg RNA was added into a 40 μl reaction mixture containing 2 μl 500 μg/ml Oligo(dT)₁₈, 2 μl dNTP mix (10 mM each), 8 μl 5× first-strand buffer, 4 μl 0.1 M DTT, 2 μl RNaseOUT™ (40 units/μl) and 2 μl SuperScript II reverse transcriptase (200 units/μl). The reaction mixture was incubated at 42° C. for 50 min and then heated at 70° C. for 15 min to terminate the reaction.

RT-PCR amplification was performed with SYBR green dye using a Mastercycler ep Realplex 4S (Eppendorf). For each run, 20 μl 2.5×Real Master Mix (Qiagen), 0.3 μM of forward and reverse primers along with 2 μl first strand cDNA were mixed. The thermal cycling conditions included an initial denaturation step at 95° C. for 2 min, 50 cycles at 95° C. for 15 s, 55° C. for 15 s and 72° C. for 20 s. Each reaction was performed in duplicates. The efficiency of the amplification was close to 2.0 (i.e. 100%) for all primer pairs. Relative expression level of each gene was calculated using the Livak method as described previously (see, Livak et al. Methods 2001, 25:402-408.) with 18S ribosomal RNA as the internal control gene. The primer sequences used were designed by Beacon Designer 5.0 (Premier Biosoft International, Palo Alto, Calif.) as shown in Table 1.

TABLE 1
Forward and reverse primer probes used for
RT-PCR of mitochondrial dehydrogenases,
pyruvate carboxylase, and glutaminase^{A}

| Gene/ | | |
|---|---|---|
| accession no | Forward sequence | Reverse sequence |
| FH/ | TCTGGTCCTCGGTCAGGTC | GACAGTGACAGCAACAT |
| NM_000143.2 | TG | GGTTCC |
| GLS/ | GCACAGACATGGTTGGTAT | AGAAGTCATACATGCCAC |
| NM_014905 | ATTAG | AGG |
| IDH3/ | CAACTGCCCCTTCTCCTAT | AGCCCAAGCCTAAGCCCA |
| NM_005530.2 | CCC | AG |
| MDH2/ | CGGAGGTGGTCAAGGCTA | CAGCGGTGTGGAGAAGT |
| NM_005918.2 | AAG | AGG |
| OGDH/ | GTGAGAATGGCGTGGACT | CGATTGATCCTGCGGTGA |
| NM_002541.2 | AC | TAC |
| PC/ | GCGTGTTTGACTACAGTGA | TCTTGACCTCCTTGAACT |
| NM_022172 | G | TG |
| SDH/ | CATCGCATAAGAGCAAAG | CCTTCCGTAATGAGACAA |
| NM_004168.2 | AAC | CC |
| 18S/ | ATCAGATACCGTCGTAGTT | CCGTCAATT |
| NR_003286 | CC | CCTTTAAGTTTCAG |

^{A}PC: pyruvate carboxylase; GLS: glutaminase; IDH3: isocitrate dehydrogenase; OGDH: α-ketoglutarate dehydrogenase; FH: fumarate hydratase; MDH2: mitochondrial malate dehydrogenase.

Western Blotting of Pyruvate Carboxylase

Pulverized and lyophilized lung tissues were extracted twice in chloroform:methanol (2:1) plus 1 mM butylated hydroxytoluene to remove lipids, which interfered with the extraction of PC. The delipidated tissue powder was then extracted in 62.5 mM Tris-HCl plus 2% sodium dodecyl sulfate (SDS) and 5 mM dithiothreitol and heated at 95° C. for 10 min. to denature proteins. The protein extract was analyzed by SDS-PAGE using a 10% polyacrylamide gel and separated proteins were transferred to a PVDF membrane (Immobilon™-P, Millipore, Bedford, Mass.), and blotted against an anti-PC rabbit polyclonal antibody (Santa Cruz Biotechnology, Santa Cruz, Calif.) overnight at 4° C. PC was visualized with incubation in a secondary anti-rabbit antibody linked to horseradish peroxidase (HRP) (Thermo Scientific, Rockford, Ill.), followed by reaction with chemiluminescent HRP substrates (Supersignal® West Dura Extended Duration substrate, Thermo Scientific), and exposure to X-ray film. The film was digitized using a high-resolution scanner and the image density of appropriate bands (130 kDa for PC and 50 kDa for α-tubulin) was analyzed using Image J (NIH, Bethesda, Md.). The image density of the PC protein band was normalized to that of the α-tubulin protein band.

Statistical Analysis

GC-MS analysis was performed in triplicate and % RSD (relative standard deviation) of analysis was calculated for each reported metabolite (Table 4). The paired t-test was performed on each pair of tumor and non-cancerous tissues for the real-time PCR data (Table 6) and for the ¹H-TOCSY data (Table 3). Linear regression calculations were done for pairs of metabolites in Table 5.

Time Course Analysis of ¹³C-Metabolites in Plasma

In order to determine the optimal time to sample tissue after infusing [U-¹³C]-Glc into lung cancer patients, we used ¹H NMR to analyze the profiles of ¹³C-labeled products in plasma samples at several time points. Other than the administered tracer itself ([U-¹³C]-Glc), the major ¹³C-labeled metabolite in plasma was lactate. Plasma samples were collected 0, 3, and 12 hr after [U-¹³C]-Glc infusion, as described in the Patient Treatment and Sample Collection section above. Unlabeled glucose and lactate concentrations were quantified by 1-D ¹H NMR using DSS as the calibration standard. Their % ¹³C enrichment was determined from the respective ¹³C satellite peaks in 1-D ¹H NMR spectra. Panels A-D illustrate the time course changes in glucose and lactate concentrations as well as their % ¹³C enrichment. Panel E shows the ¹³C satellite pattern of the 3-methyl group of lactate (¹³C-CH₃-lac) in the 1-D ¹H NMR spectrum of patient #6 after 3 hr of [U-¹³C-Glc] infusion. The chemical shifts of lactate and Ala reflected the acidic pH of the TCA extract.

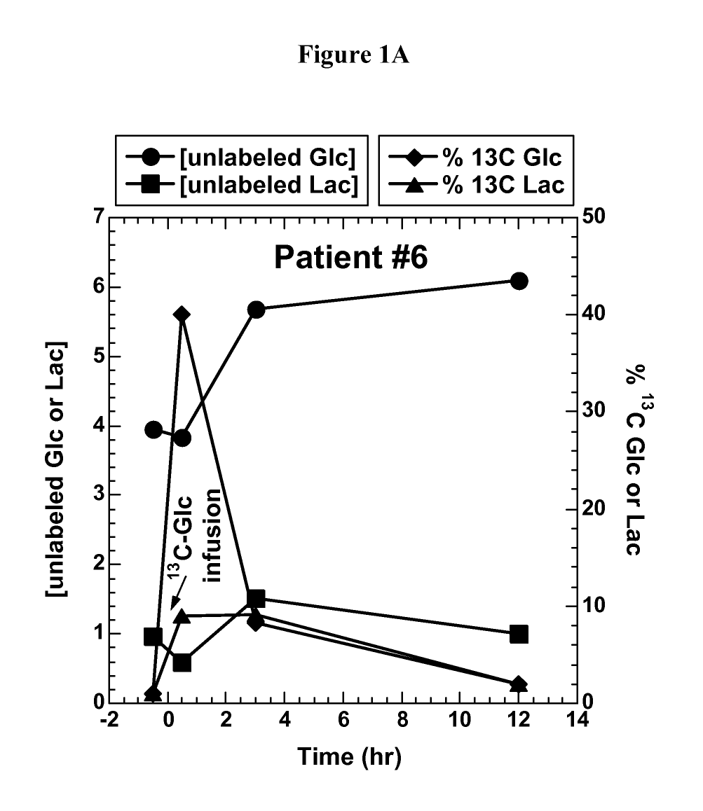

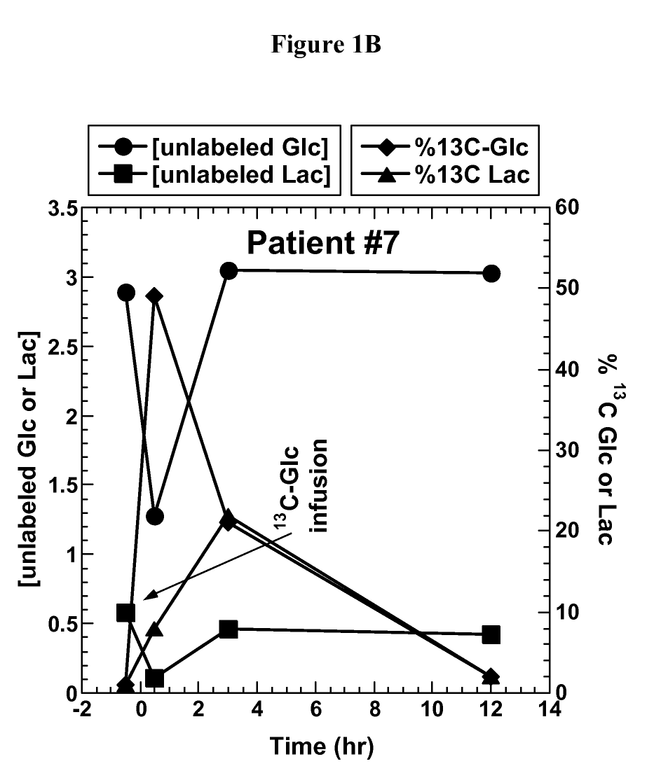

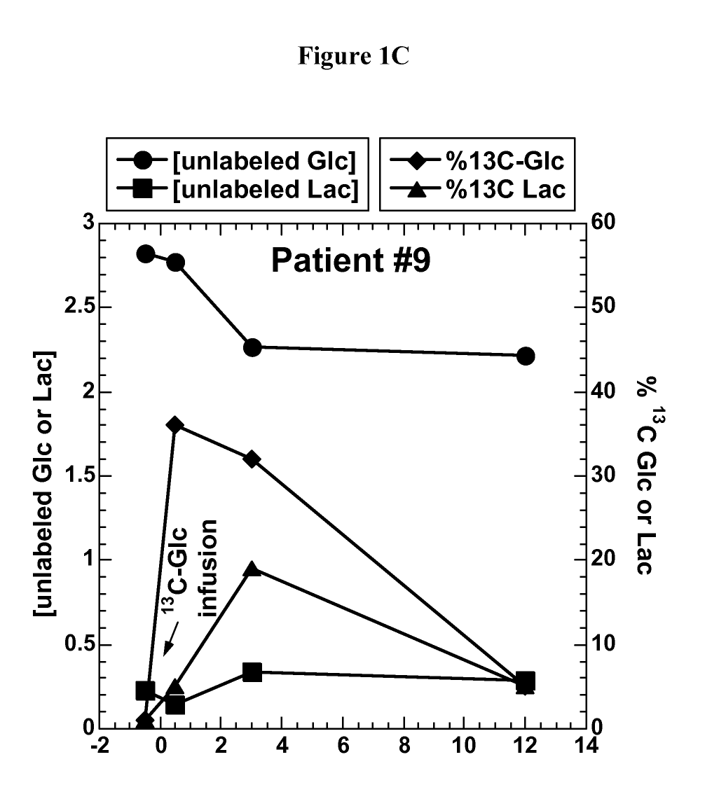

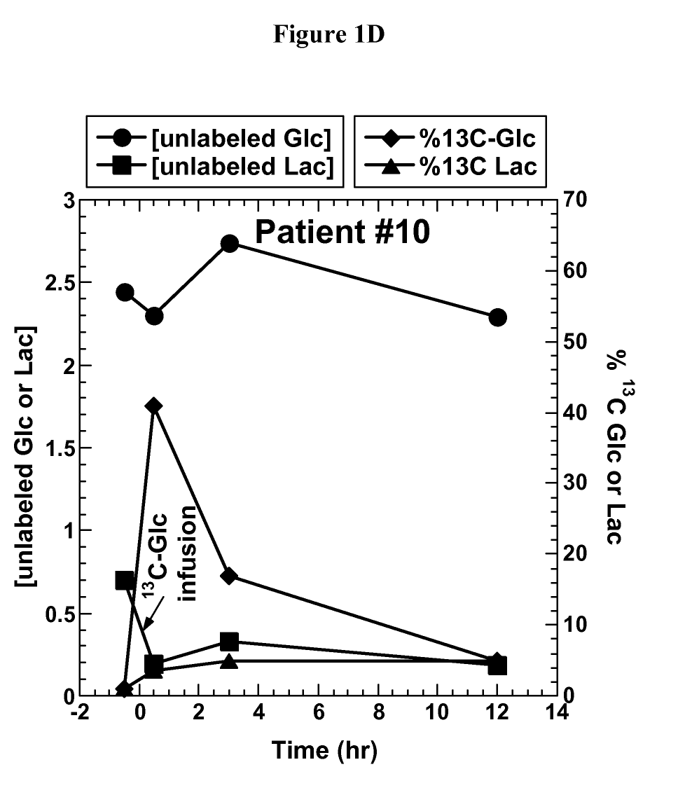

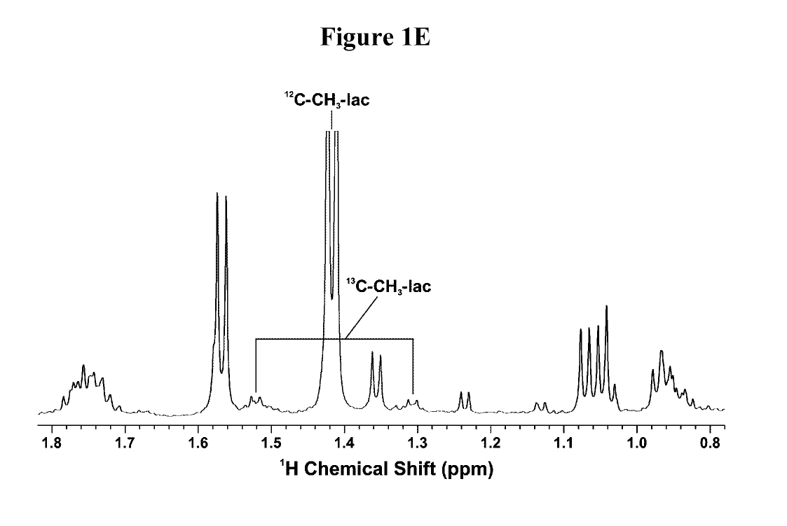

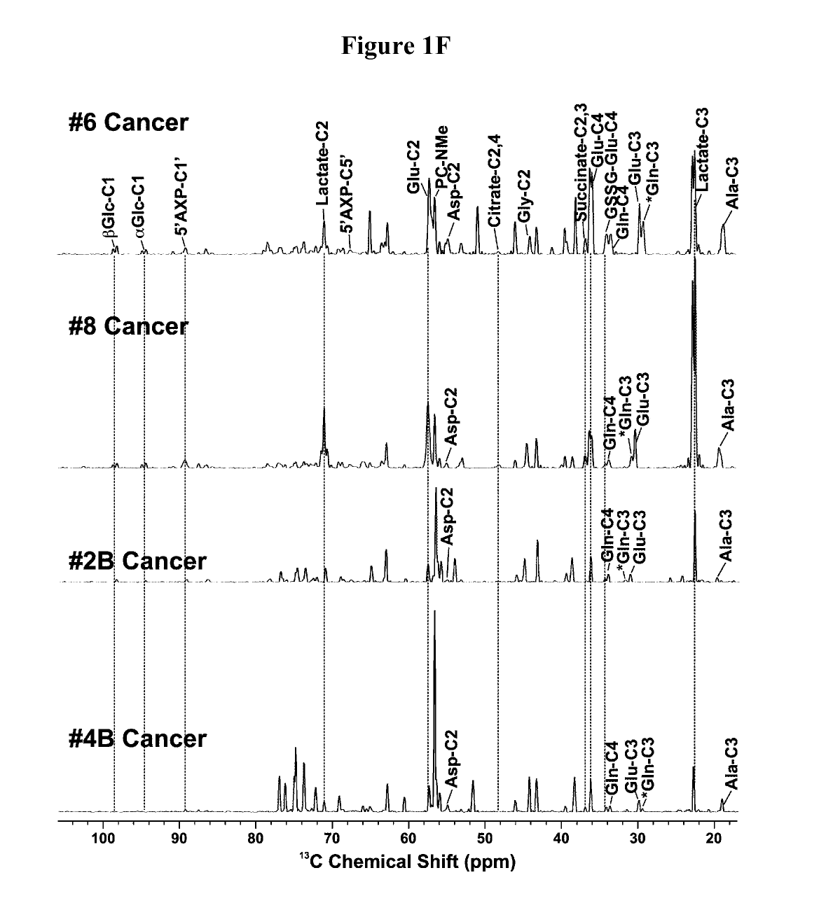

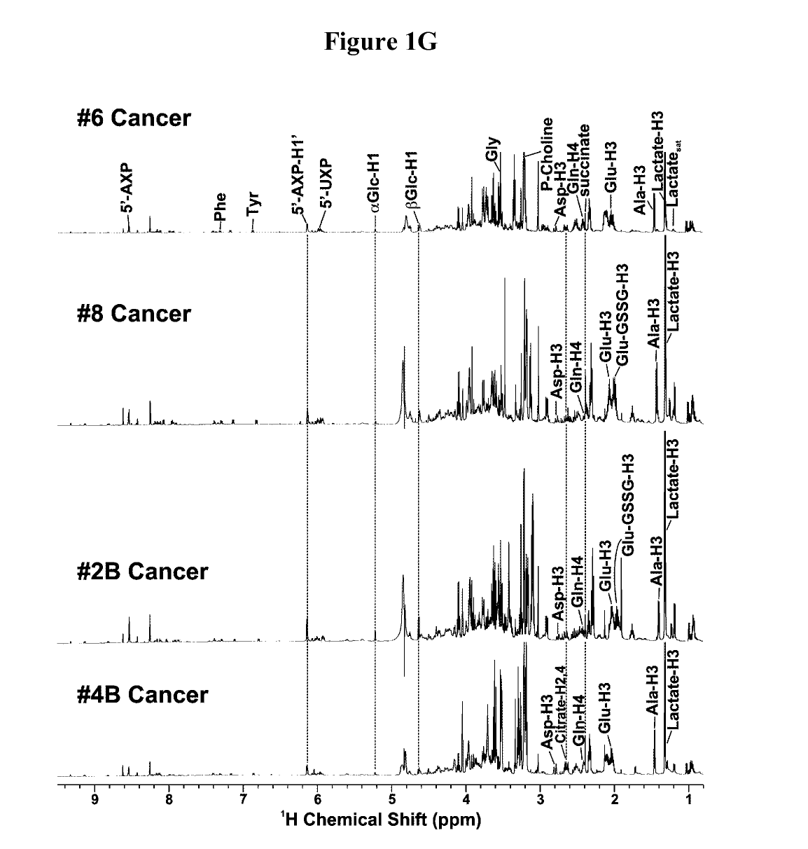

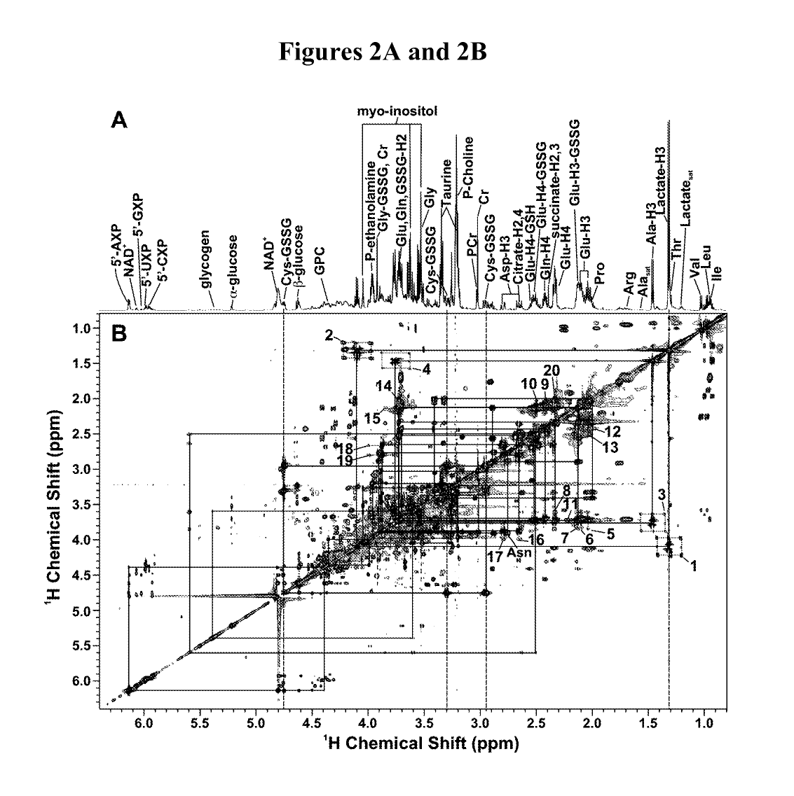

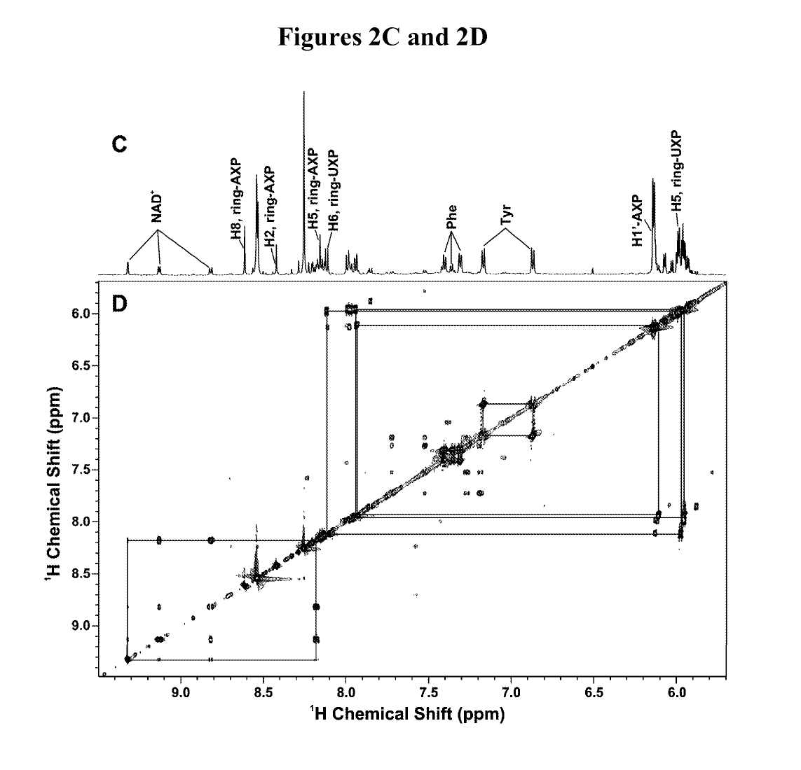

As shown in FIG. 1A-1D, plasma glucose was maximally enriched (up to 49%) in ¹³C immediately (0.5 hr) following the [U-¹³C]-glucose infusion. Three hours later, a significant fraction (8.3-32%) of the plasma glucose remained ¹³C-labeled but by 12 hrs, the % ¹³C enrichment dropped to 2-5%, as illustrated for four patients. The % ¹³C-labeled lactate showed a similar time course as the % ¹³C-labeled glucose, except that the % enrichment reached a maximum (5-22%) after 3 hrs of infusion (FIGS. 1A-1D). These data show that once taken up by tissues, ¹³C-glucose was metabolized quickly to ¹³C-lactate and secreted back into the blood. In addition, a large fraction of the plasma ¹³C-lactate was uniformly labeled in ¹³C, as evidenced by the fine splitting structure of the ¹³C satellites of the 3-methyl protons of lactate (FIGS. 1E and 2) and by GC-MS analysis (See Table 2).

TABLE 2
Percentage distribution of ¹³C-mass isotopomers of lactate
in the plasma of human lung cancer patients infused with
[U-¹³C]-glucose for 3 h^{A}

| | lactate + 1^{B} | lactate + 2^{B} | lactate + 3^{B} |
|---|---|---|---|
| #6 | 0.80% | 0.36% | 1.89% |
| #7 | 0.00% | 1.83% | 4.84% |
| #8 | 2.94% | 0.16% | 3.31% |
| #9 | 0.00% | 4.11% | 3.47% |
| #10 | 2.01% | 1.00% | 6.37% |

^{A}Values in excess of natural abundance as determined from GC-MS analysis;
^{B}Lactate + 1, lactate + 2, lactate + 3 refer to lactate with one, two, or three ¹³C-labeled carbons, respectively.

GC-MS analysis also revealed a significant presence of mass isotopomers of lactate with one or two ¹³C labels for patients #8-10, which is consistent with an active Cori cycle. Based on this time-course analysis of ¹³C-isotopomers of metabolites in human plasma samples, we chose a duration of 3-4 h between ¹³C-glucose infusion and surgical resection for patients #6-10 in order to optimize ¹³C incorporation from [U-¹³C]-Glc into various metabolites.

Of the twelve subjects undergoing ¹³C glucose infusion, the median age was 63 (range 52-76), 67% were male, 67% had squamous cell carcinoma, and 33% adeno- or adenosquamous carcinoma, all of grade II or III. All subjects had a history of heavy smoking.

Detection of Selective ¹³C Enrichment in Specific Carbon Positions of Lung Tissue Metabolites

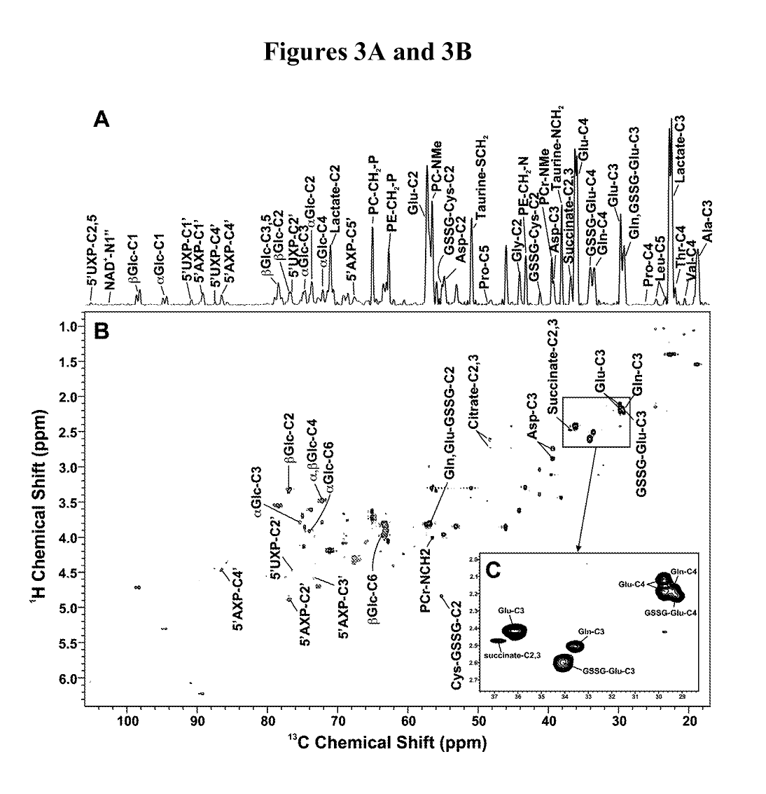

Comparison of metabolite profiles in TCA extracts of paired normal and cancerous lung tissues of patient #6. Metabolites in the ¹³C HSQC projection (panel F) and 1-D ¹H NMR spectra (panel G) were assigned as in FIGS. 2 and 3, respectively. The two sets of spectra were normalized to dry weight and spectral parameters such that the peak intensity of individual resonances is directly comparable. The dashed lines trace metabolites that differed in abundance between cancerous and normal lung tissues.

FIG. 1F compares the HSQC projection spectra along the ¹³C dimension (FIG. 1F) with the 1-D ¹H NMR spectra (FIG. 1G) for two each patients infused with [U-¹³C]-Glc (patients #6 and 8) or without (patients #2B and 4B). It should be emphasized that for patients #2B and 4B, the ¹³C resonances in FIG. 1F arose from natural abundance only (ca. 1.1% of total concentration), while some of the resonances for patients #6 and 8 were enriched due to ¹³C label incorporation from [U-¹³C]-Glc, e.g. the 3-carbon of lactate (Lactate-C3).

Selective ¹³C enrichment over natural abundance was revealed as follows. For example, in the ¹H spectra (FIG. 1G), the peak intensity of the methyl protons attached to the unlabeled 3-carbons of Ala (Ala-H3) and Asp (Asp-H3) was comparable for all four patients, which indicates that the unlabeled Ala and Asp concentrations were comparable. In contrast, the ¹³C peak intensity (or abundance) of the 3-carbon of Ala (Ala-C3) as well as 2 and 3-carbon of Asp (Asp-C2, Asp-C3), FIG. 1F) was proportionally higher for [U-¹³C]-Glc-infused patients (#6 and #8) than for patients without the ¹³C infusion (#2B and #4B). If ¹³C intensities in HSQC spectra were attributed to natural abundance only, then one would expect them to be similar for Ala-C3 and Asp-C2 since Ala and Asp concentrations were comparable. The higher ¹³C abundance of Ala-C3 and Asp-C2 is thus consistent with ¹³C enrichment in these two carbons over the natural abundance for patients #6 and #8.

A similar reasoning applies to the ¹³C enrichment in the 2,3 carbons of succinate (succinate-C2,3), 2,4 carbons of citrate and the 1′-carbon of the ribosyl residue of adenine nucleotides (5′AXP-C1') in patient #6. The unlabeled succinate, citrate and AXP levels in #6 were lower than those in #2B and 4B (as estimated from the intensities of 5′AXP-H1′, citrate and succinate ¹H resonances in FIG. 1G). However, the intensity of the ¹³C resonances for 5′AXP-C1, citrate-C2,4 and succinate-C2,3 in FIG. 1F showed an opposite trend. This pattern of lower concentrations yet higher ¹³C abundance or selective enrichment for Patient #6—interpretable as higher turnover of 5′-AXP, citrate, and succinate—reiterates the point that steady-state concentrations do not necessarily directly track metabolic activity.

In addition, the ¹³C enrichment in the C-1 carbons of α- and β-glucose was clearly demonstrated by the higher intensity and doublet nature of the ¹³C resonances (due to ¹³C-1 and ¹³C-2 spin coupling), in contrast to the lower intensity singlet for #2B and 4B (FIG. 1F). It should be noted that these isotopomers were not evident in the TOCSY spectrum for #6 (FIG. 2) due to the lack of detectable ¹³C satellite cross-peaks. Furthermore, the higher intensity doublet (due to ¹³C-3 and ¹³C-2 spin coupling) of the C-3 resonance of lactate (lactate-C3) for #6 and 8 relative to the less intense singlet for #2B and 4B (FIG. 1F) indicate ¹³C labeling at both 3- and 2-carbons of lactate. The triplet shape of the lactate-C2 resonance is consistent with ¹³C spin coupling of this carbon to those at the 1 and 3 positions. This result verified the presence of [U-¹³C]-lactate, as indicated by the TOCSY (See, FIG. 2B) and GC-MS analysis (See, Table 4). Finally, the more intense and doublet appearance of Glu-C4 for #6 and 8 indicate ¹³C labeling at this carbon along with C-5 or C-3 of Glu.

Metabolite and ¹³C-Isotopomer Profiling of Lung Tissue Extracts by NMR

An example of the assignment of metabolites and their ¹³C-isotopomers in the TCA extracts by 2-D ¹H TOCSY and high-resolution 1-D NMR spectra is illustrated in FIG. 2. A 2-D ¹H TOCSY identification of metabolites in the lung tumor tissue of patient #6 is displayed along with the corresponding 1-D high-resolution spectrum. Panels 2A, 2B and 2C, 2D show the 0.8-6.4 and 5.7-9.5 ppm regions of the spectra, respectively. The assignment of cystine residue of oxidized glutathione (GSSG) was illustrated, which was based on the ¹H covalent connectivity (traced by solid rectangles) and chemical shifts (traced by dashed lines). Lactate was discerned similarly and based on the peak splitting pattern (doublet for 3-methyl @ 1.32 ppm and quartet for 2-methine protons @ 4.11 ppm). In addition, the ¹³C satellite cross-peaks of 3-methyl and 2-methine protons of lactate (patterns 1 and 2) and Ala (patterns 3 and 4) were evident (traced by dashed rectangles), and the peak pattern indicates that lactate and Ala were uniformly ¹³C labeled. The ¹³C satellite cross-peak patterns for the protons of Glu (5, 6, 12, 20), Gln (9), glutamyl residue of oxidized glutathione (GSSG) (7, 10, 13) and Asp (16-19) were present and noted by vertical and horizontal dashed lines. The ¹³C satellite cross-peaks 14 and 15 were contributed by a mixture of ¹³C-2-Glu, ¹³C-2-Gln, and ¹³C-2-Glu of reduced glutathione (GSH). Assignments were made by matching the chemical shifts, spin-spin coupling patterns, and available covalent connectivities (traced by solid rectangles in the TOCSY contour maps) of individual resonances against those of the standard compounds (Fan et al., Progress in NMR Spectroscopy 2008, 52:69-117; Fan, Progress in Nuclear Magnetic Resonance Spectroscopy 1996, 28:161-219).

Some metabolites observed in lung tissue extracts include isoleucine (Ile), leucine (Leu), valine (Val), lactate, alanine (Ala), arginine (Arg), proline (Pro), glutamate (Glu), oxidized glutathione (GSSG), glutamine (Gln), succinate, citrate, aspartate (Asp), creatine (Cr), phosphocholine (P-choline), taurine, glycine (Gly), phenylalanine (Phe), tyrosine (Tyr), myo-inositol, α- and β-glucose, NAD⁺, cytosine nucleotides (CXP), uracil nucleotides (UXP), guanine nucleotides (GXP), and adenine nucleotides (AXP). The ¹H TOCSY assignment of metabolites was complemented by the 2-D ¹H-¹³C HSQC analysis of the same extract, as shown in FIG. 3, where the ¹H-¹³C covalent bonding patterns were observed. The HSQC spectrum provided better resolution for some metabolites such as Glu, Gln, and GSSG (FIG. 3C, inset), thereby confirming their assignment.

In addition to metabolite identification, the TOCSY and HSQC analyses provided ¹³C positional isotopomer information for several metabolites in the lung TCA extracts. The ¹H TOCSY data (See, FIG. 2B) unambiguously revealed the presence of uniformly ¹³C labeled lactate ([U-¹³C]-lactate) and Ala (([U-¹³C]-Ala) by the ¹³C satellite cross-peak pattern (patterns 1-4 [symbol] ) of 3-methyl and 2-methine protons of lactate (lactate-H3 and H2) and Ala (Ala-H3 and H2) (traced by dashed rectangles in FIG. 2B). This pattern is consistent with the enrichment of multiply labeled lactate and alanine of 200-300 fold over the natural abundance levels. The presence of ¹³C-3-Glu, ¹³C-3-Gln, and ¹³C-3-glutamyl residue of oxidized glutathione (Glu-GSSG) was evidenced by the ¹³C satellite cross-peak patterns 9, 10, 14 ( [symbol] ) and 11-13 () (traced by dashed lines in FIG. 2B). Patterns 5-8 and 15 denote the presence of ¹³C-2-Glu-GSSG and ¹³C-2-Glu while patterns 16 and 17 indicate the presence of ¹³C-2-Asp.

FIG. 3 shows ¹H-¹³C 2-D HSQC identification of ¹³C-metabolites in the TCA extracts of lung tumor tissues of patient #6. Metabolites were identified based on ¹H-¹³C covalent linkages observed in the 2-D contour map (panel B) and from the TOCSY spectrum such as in FIG. 2B. Panel A is the 1-D projection spectrum of the 2-D data along the ¹³C dimension, which allows a better comparison of the peak intensity of different metabolites. Panel C displays the expanded spectral region of C3 and C4 resonances of Glu, Gln, and GSSG-Glu to illustrate the resolution of these resonances in the 2-D HSQC contour map.

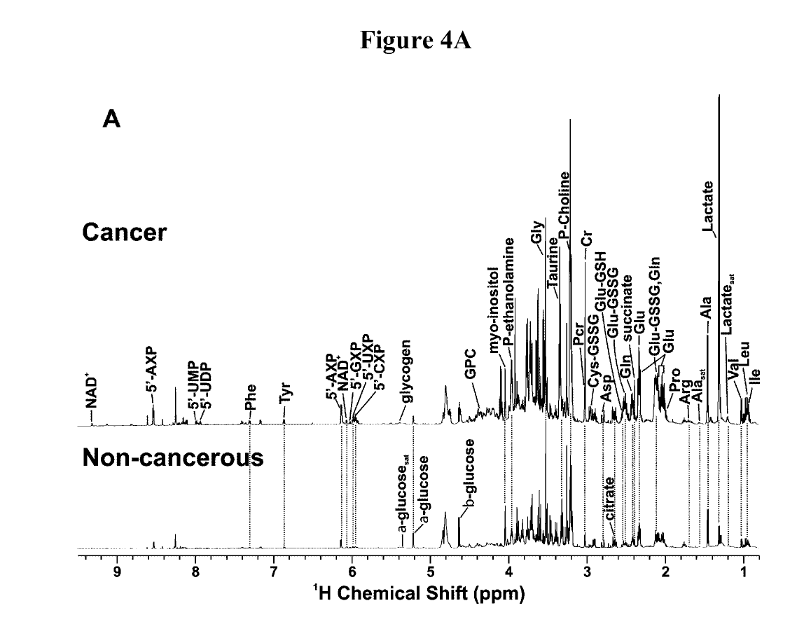

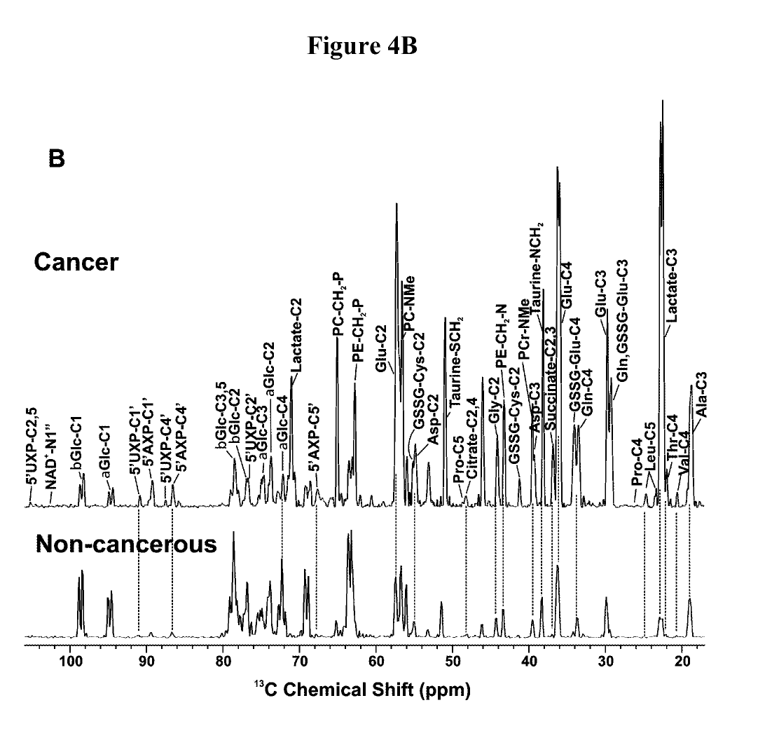

FIG. 4 displays a comparison of metabolite profiles in TCA extracts of paired non-cancerous and cancerous lung tissues of patient #6. Metabolites in the 1-D ¹H NMR (panel A) and ¹³C HSQC projection spectra (panel B) were assigned as in FIGS. 2 and 3, respectively. The two sets of spectra were normalized to dry weight and spectral parameters such that the peak intensity of individual resonances is directly comparable. The dashed lines trace metabolites that differed in abundance between cancerous and non-cancerous lung tissues.

The ¹³C-positional isotopomer information obtained from the TOCSY analysis was confirmed by the HSQC analysis, including the positional isotopomers of ¹³C-3-Ala, ¹³C-3-lactate, ¹³C-3-Glu, ¹³C-3-Gln ¹³C-3-Glu-GSSG, ¹³C-2-Glu-GSSG, ¹³C-2-Glu, and ¹³C-2-Asp (See, respectively Ala-C3, Lactate-C3, Glu-C3, Gln-C3, GSSG-Glu-C3, Glu-GSSG-C2, Glu-C2, and Asp-C2 in FIGS. 3B and 4B). In addition, the HSQC data also provided complementary information on isotopomers whose ¹³C satellite cross-peaks were too weak to observe or masked by other cross-peaks in the crowded regions of the TOCSY spectrum (See, e.g., Detection of Selective ¹³C Enrichment in Specific Carbon Positions of Lung Tissue Metabolites section). These included selective ¹³C enrichment in C-3-Asp, C-2,3-succinate, C-2,4-citrate, C-1′-ribose-5′AXP, and C-1-α- and -β-glucose (See, respectively Asp-C3, succinate-C2,3, citrate-C2,4,5′AXP-C1', and α- and β-Glc-C1, FIGS. 3B and 4B).

Based on the metabolite assignment in FIGS. 2 and 3, the 1-D ¹H NMR and ¹³C HSQC projection spectra allowed comparison of metabolite and ¹³C isotopomer profiles in the TCA extracts of paired non-cancerous and cancerous lung tissues. This is illustrated in FIG. 4 for patient #6. It is clear in FIG. 4A that the majority of the metabolites (except for glucose) were present at a higher level in the cancerous than in the non-cancerous lung tissue. These also included the ¹³C-labeled isotopomers, [U-¹³C]-lactate and [U-¹³C]-Ala (as denoted respectively by the lactateₛₐₜ and Alaₛₐₜ resonance in FIG. 4A). The extent of ¹³C enrichment in lactate, Ala (as uniformly labeled species) and Glu (at the C-2 position) was quantified from the respective 2-D ¹H TOCSY cross-peak patterns (See, FIG. 2B), as previously described. They were consistently higher in the cancer than in the non-cancerous tissue from the same patient, as shown in Table 3.

TABLE 3
Enhanced ¹³C enrichment of metabolites in lung cancer
tissues relative to their normal counterpart

| | | | % [U-¹³C]- | | % [U-¹³C]- | | | |
|---|---|---|---|---|---|---|---|---|
| | % [U-¹³C]-Ala^{A} | | Lactate^{A} | | Glucose^{A} | | % [¹³C-2]-Glu^{A} | |
| Compound | Normal | Cancer | Normal | Cancer | Normal | Cancer | Normal | Cancer |
| Mean | 1.8 | 9.1 | 8.6 | 15.1 | 15.5 | ND^{B} | 5.4 | 8.3 |
| SD^{C} | 0.9 | 4.9 | 4.6 | 5.4 | 2.3 | — | 1.1 | 1.9 |

| | | | | |
|---|---|---|---|---|
| p value^{D} | 0.009 | 0.02 | — | 0.012 |

^{A}Values are average of seven patients; % ¹³C-Ala, lactate, and Glu were determined from 2-D ¹H TOCSY data using appropriate cross-peaks; % [U-¹³C]-glucose was obtained from 1-D ¹H spectra;
^{B}Not detected or below detection limit;
^{C}Standard deviation;
^{D}from paired t-test.

The extent of ¹³C enrichment in glucose (as [U-¹³C]-Glc) for non-cancerous tissues was determined from the 1-D ¹H spectra (See, FIG. 4A), which was considerably lower in the cancer than in its non-cancerous counterpart (See, Table 3). Moreover, increased ¹³C abundance (or ¹³C peak intensity) of most metabolites in cancer relative to non-cancerous tissues was evident in the 1-D HSQC projection spectra in FIG. 4B. Part of the increase in ¹³C abundance from non-cancerous to cancer tissues reflected the difference in total metabolite concentration, which contains 1.1%¹³C at natural abundance. However, selective enrichment in ¹³C for a number of metabolite carbons also contributed to the increase in their ¹³C peak intensity (see, e.g., Detection of Selective ¹³C Enrichment in Specific Carbon Positions of Lung Tissue Metabolites section). These include C-3-Ala, C-2,3-lactate, C-3-Gln+GSSG, C-2 to 4-Glu, C-4-Gln, C-4-GSSG, C-2,4-citrate, C-2,3-Asp, C-1′,4′,5′-5′-AXP, and C-1′,4′-5′-UXP.

To quantify the ¹³C abundance of metabolites at specific carbon positions, the 2-D HSQC spectra were utilized for the better resolution than the 1-D ¹³C projection spectra (See, FIG. 4B). This was performed for ¹³C-2,3-succinate of patients #6-10 by integrating the volume of its HSQC cross-peak (See, FIG. 3C). The relative ¹³C abundance (a measure of selective enrichment) of these two carbons was calculated by normalizing the HSQC peak volume to the total succinate concentration. The relative ¹³C abundance of C-2,3-succinate for tumor tissues (3.6±1.2) was significantly greater than that for non-cancerous tissues (0.6±0.7) with a p value of <0.01.

Metabolite and ¹³C-Isotopomer Profiling of Lung Tissue Extracts by GC-MS

Parallel analysis of the lung TCA extracts by GC-MS served to verify key findings in the metabolite profile obtained by NMR while providing absolute quantification of a subset of metabolites and their ¹³C mass isotopomers. Table 4 shows the quantification of selected metabolites and their total ¹³C enrichment (in excess of natural abundance) by GC-MS. Also shown is the quantification of the m+3 mass isotopomer of Asp (¹³C₃-Asp or Asp with three of its carbons labeled in ¹³C).

TABLE 4
Total Metabolite and ¹³C-enriched metabolite content of paired normal
and cancerous lung tissues from [U-¹³C]-glucose administered patients ^{A}

| | #6 | | #7 | | #8 | | #9 | | #10 | |
|---|---|---|---|---|---|---|---|---|---|---|
| | sqC ^{B}, grade II | | adenoC ^{B}, grade II | | sqC ^{B}, grade II-III | | sqC ^{B}, grade II | | sqC ^{B}, grade III | |
| Compound | N ^{B} | C ^{B} | N | C | N | C | N | C | N | C |
| Total Ala | 4.67 | 15.76 | 6.25 | 6.44 | 4.97 | 9.28 | 5.14 | 15.08 | 5.42 | 27.34 |
| ¹³C-Ala ^{C} | 0.03 | 0.28 | 0.15 | 0.34 | 0.09 | 0.57 | 0.21 | 0.79 | 0.29 | 1.60 |
| Total Asp | 1.32 | 4.27 | 3.83 | 3.09 | 2.67 | 1.51 | 1.94 | 4.78 | 2.27 | 1.66 |
| ¹³C-Asp ^{C} | 0.18 | 0.59 | 0.36 | 0.44 | 0.35 | 0.24 | 0.21 | 0.82 | 0.36 | 0.31 |
| ¹³C₃-Asp ^{C, D} | <0.004 | 0.058 | 0.009 | 0.005 | 0.006 | 0.009 | <0.004 | 0.024 | 0.004 | 0.020 |
| | (<0.3%) | (1.4%) | (0.2%) | (0.16%) | (0.2%) | (0.6%) | (<0.2%) | (0.5%) | (0.17%) | (1.2%) |
| Total Cit ^{B} | 0.60 | 1.32 | 1.21 | 1.18 | 1.28 | 1.56 | 0.77 | 1.53 | 0.74 | 1.37 |
| ¹³C-Cit ^{B, C} | 0.03 | 0.10 | 0.02 | 0.05 | 0.08 | 0.12 | 0.03 | 0.11 | <0.004 | 0.12 |
| Total Glu | 0.24 | 4.80 | 2.96 | 2.97 | 1.88 | 3.25 | 1.91 | 4.78 | 1.82 | 3.28 |
| ¹³C-Glu ^{C} | 0.03 | 0.16 | <0.004 | <0.004 | <0.004 | 0.22 | <0.004 | 0.36 | 0.10 | 0.36 |
| Total Gln | 0.56 | 7.68 | 1.41 | 1.02 | 0.64 | 1.32 | 0.74 | 2.71 | 0.79 | 2.99 |
| ¹³C-Gln ^{C} | 0.07 | 0.34 | 0.07 | 0.00 | 0.04 | 0.00 | 0.00 | 0.21 | 0.00 | 0.04 |
| Total Lac ^{B} | 2.66 | 29.55 | 11.80 | 10.62 | 9.44 | 17.72 | 19.52 | 25.31 | 8.27 | 29.67 |
| ¹³C-Lac ^{C} | <0.004 | 0.66 | 0.19 | 0.63 | 0.19 | 0.88 | <0.004 | 1.44 | 0.03 | 0.94 |
| Total Succ ^{B} | 0.17 | 1.34 | 0.50 | 0.70 | 0.89 | 1.84 | 0.64 | 1.59 | 1.14 | 3.13 |
| ¹³C-Succ ^{C} | 0.02 | 0.08 | 0.01 | 0.03 | 0.03 | 0.11 | 0.08 | 0.14 | 0.06 | 0.19 |

^{A} in μmole/g dry weight as determined by GC-MS; the % RSD was generally <3% in triplicate analyses with the exception of Gln (11%) and ¹³C₃-Asp (27%);
^{B} sqC: squamous cell carcinoma; adenoC: adenocarcinoma; N: normal; C: cancer; Cit: citrate; Lac: lactate; Succ: succinate;
^{C} ¹³C enrichment in excess of natural abundance; values in bold represent enhanced enrichment in cancer over its normal counterpart;
^{D} ¹³C mass isotopomer of Asp with three carbons labeled; detection limit was 0.004 μmole/g dry weight. Values in parentheses are the percentages of ¹³C₃ isotopomers of the total aspartate. This was much greater than 5 × 10⁻⁴% at natural abundance.

For patient #6, the GC-MS data revealed excess total ¹³C enrichment in tumor over non-cancerous tissues for Ala, Asp, Glu, Gln, lactate, citrate, and succinate. An enhanced production of ¹³C₃-Asp in the tumor compared with the paired non-cancerous tissue was also evident. This is consistent with the NMR observation (See, FIG. 4B) of the buildup of various ¹³C positional isotopomers of metabolites including [¹³C-3]-Ala, [¹³C-2,3]-lactate, [¹³C-2,3,4]-Glu, [¹³C-4]-Gln, [¹³C-2,3]-Asp, [¹³C-2,4]-citrate, and [¹³C2,3]-succinate for the tumor, compared with the paired non-cancerous tissues of #6. A similar ¹³C enrichment pattern of Ala, lactate, succinate, and citrate in lung tumor tissues was observed in all five patients where ¹³C labeling in these metabolites was sufficient to be quantified by GC-MS. In addition, the enhanced synthesis of ¹³C₃-Asp in tumor tissues was evident in four of the five patients (Table 4). It should be noted that the fraction of ¹³C₃-Asp in tumor tissues (0.5 to 1.4%) was above the natural abundance background (5×10⁻⁴%) (e.g., Table 4).

Quantitative Correlation of ¹³C-Metabolites in Human Lung Tissues

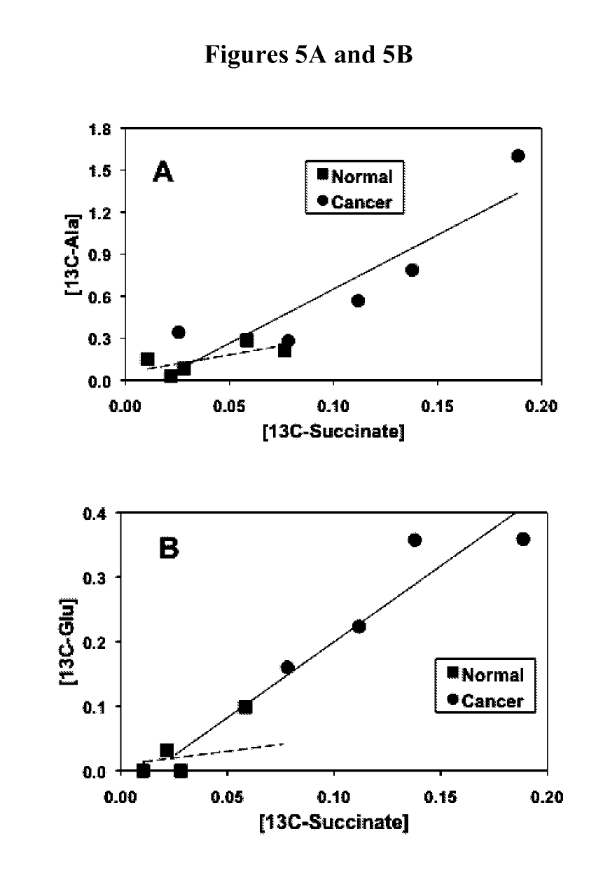

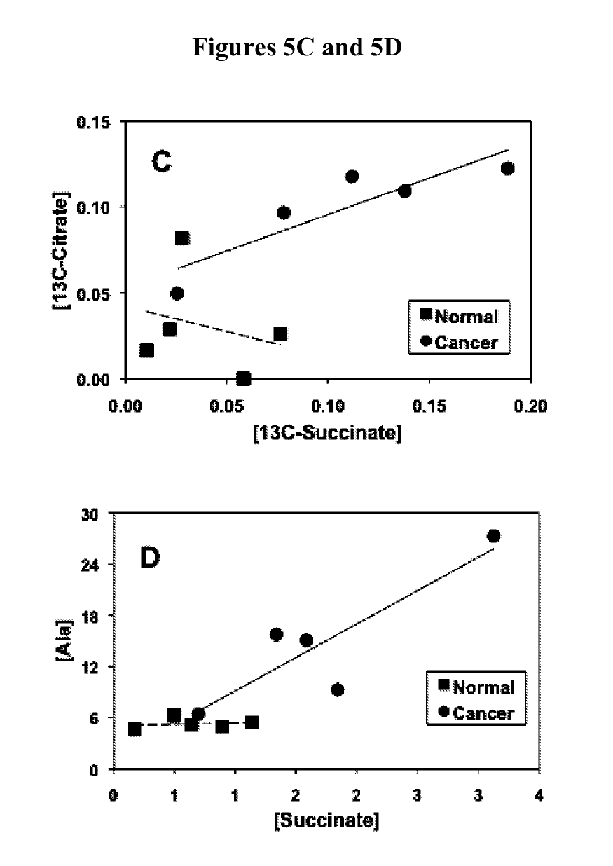

Utilizing the GC-MS data, pairs of biosynthetically-related ¹³C-labeled metabolites in tumor and non-cancerous tissues were tested for precursor-product relationships, as illustrated in FIG. 5 and Table 5.

FIG. 5 shows the relationships between Krebs cycle intermediates and glycolytic products in terms of ¹³C-labeled and total concentrations for lung tumor and non-cancerous tissues resected from patients #6-10. Metabolite and ¹³C isotopomer concentrations ([metabolite]) were determined as described in the GC-MS analysis section above. The R² of the linear fit (solid lines for cancer and dash lines for non-cancerous tissues) for these plots are listed in Table 5.

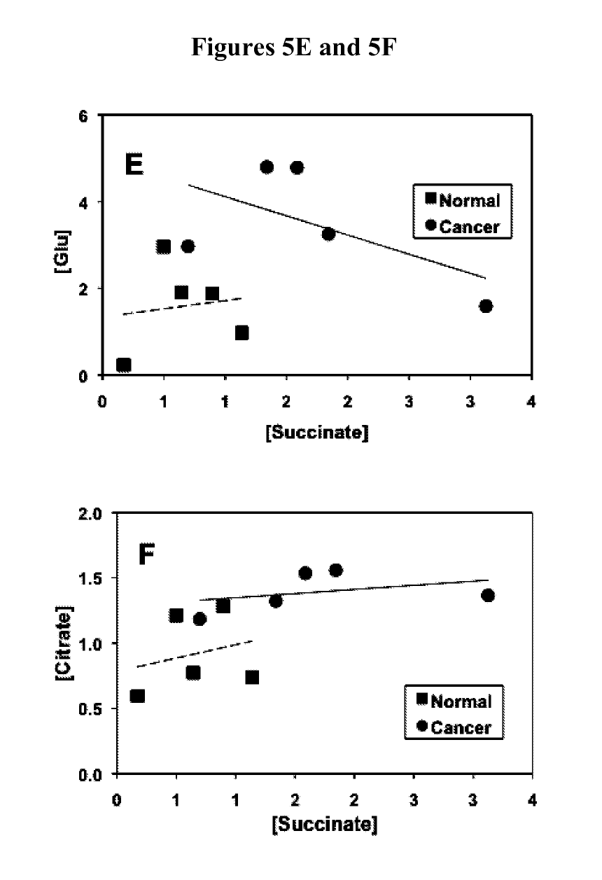

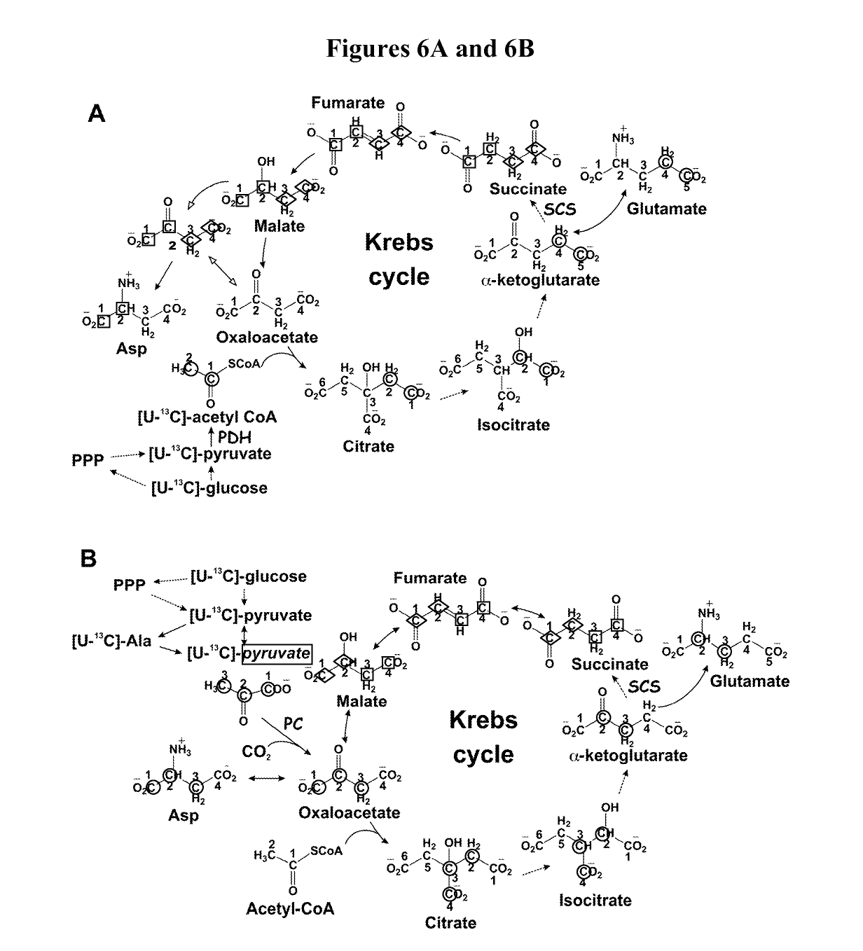

FIG. 6 displays the expected ¹³C labeling patterns in mitochondrial Krebs cycle intermediates and byproducts with [U-¹³C]-Glc as tracer. The cycle reactions are depicted without (panel A) or with (panel B) anaplerotic pyruvate carboxylase (PC) reaction and the ¹³C positional isotopomer patterns illustrated are the result of one cycle turn. In the absence of pyruvate carboxylation, Glu is labeled at C4 and C5 positions via the forward cycle reactions while Glu is labeled at C2 and C3 when pyruvate carboxylation is active (panels A and B). The possibility that a separate pool of pyruvate derived from Ala for entry into the Krebs cycle via pyruvate carboxylation is depicted in panel B, along with the contribution of the non-oxidative branch of the pentose phosphate pathway (PPP) to the pyruvate pool. Isotopic scrambling occurs at the symmetric succinate, leading to the redistribution of ¹³C labels into its four carbons, two each at a time (carbons surrounded by squares and diamonds). The letter C's surrounded by circles represent ¹³C labeled carbons before scrambling. Squares and diamond shapes around the letter C's represent ¹³C labeled carbons after scrambling. The pyruvate with a rectangle around it denotes a separate pool of pyruvate. Solid and dashed arrows denote favorable single and multi-step reactions, respectively. Open arrows in panel A delineate ¹³C-labeled OAA after one turn from unlabeled pre-existing OAA. ¹³C-succinate, ¹³C-citrate, and ¹³C-Glu are products of the Krebs cycle while ¹³C-Ala is derived from pyruvate, the end product of glycolysis (See, FIG. 6).

TABLE 5
Linear correlation for total or ¹³C-labeled concentrations
between pairs of glycolytic- and Krebs cycle-derived
metabolites in human lung tissues^{A}

R²

| Plots^{B} | Normal | Cancer |
|---|---|---|
| [Ala] versus [succinate] | 0.025 | 0.761 |
| [¹³C-Ala] versus [¹³C-succinate] | 0.483 | 0.792 |
| [lactate] versus [succinate] | 0.056 | 0.395 |
| [¹³C-lactate] versus [¹³C-succinate] | 0.412 | 0.374 |
| [Glu] versus [succinate] | 0.018 | 0.343 |
| [¹³C-Glu] versus [¹³C-succinate] | 0.072 | 0.912 |
| [citrate] versus [succinate] | 0.061 | 0.133 |
| [¹³C-citrate] versus [¹³C-succinate] | 0.069 | 0.789 |

^{A}R² for the linear regression fit was calculated using Excel for patients #6-10 (n = 5);
^{B}Obtained from GC-MS analysis; [metabolite]: total concentration in μmole/g dry weight of metabolite; [¹³C-metabolite]: μmole/g dry weight of ¹³C-labeled metabolites

A linear correlation in the concentration of ¹³C-succinate with that of ¹³C-Ala, ¹³C-Glu, or ¹³C-citrate (FIG. 5A-C, Table 5) was discernable for lung cancer tissues but not for non-cancerous lung tissues. Also noted was the less significant correlation between ¹³C-succinate and ¹³C-lactate for the lung tumor tissues (Table 5). When total concentrations were plotted, the correlation was even less apparent, particularly for the non-cancerous tissues (FIG. 5D-F, Table 5). This observation underscores the need for acquiring ¹³C-isotopomer data, instead of just steady-state concentrations, to deduce meaningful relationships between transformed products in related pathways. Moreover, in all six plots of FIG. 5, a separation of non-cancerous and tumor tissues was evident, i.e. the tumor and non-cancerous tissues clustered in the high and low concentration quadrants, respectively.

Glycolysis and Krebs Cycle in Human Lung Tumors is Activated

Based on the ¹³C isotopomer analysis by NMR (e.g. FIG. 4, Table 3) and GC-MS (Tables 4-5), human lung tumor tissues exhibited an increased capacity for carbon incorporation from glucose into lactate, Ala, citrate, Glu, succinate, ribosyl moiety of nucleotides, and Asp relative to the surrounding “non-cancerous” lung tissues. The transformation of [U-¹³C]-Glc into [U-¹³C]-lactate and [U-¹³C]-Ala can only occur via glycolysis and to a much lesser extent, the pentose phosphate pathways (PPP), whereas ¹³C-ribose of nucleotides can only be derived from PPP. The enhanced production of these metabolites in tumor tissues provided metabolic evidence for the enhancement of the glycolytic capacity. Increased production of ¹³C-ribose of nucleotides may have resulted from an enhancement in oxidative and/or non-oxidative branches of the PPP in lung tumor tissues.

The increased conversion of ¹³C carbons from glucose into the Krebs cycle intermediates (citrate and succinate) or related metabolites (Glu and Asp) in tumor compared to non-cancerous tissues (See, FIG. 4 and Tables 3-4) is unexpected. We anticipated a reduced and/or disrupted transformation of glucose-carbon into the intermediates of the Krebs cycle, based on the recognized concept of mitochondrial “dysfunction” in cancer. Instead, the cycle capacity was not reduced in any of the five lung tumor tissues analyzed. This finding is supported by the enhanced ¹³C incorporation into citrate, succinate, Glu (a surrogate marker of α-ketoglutarate or αKG) and Asp (a surrogate marker of oxaloacetate or OAA) in tumor tissues (Tables 3-5 and FIG. 4B). The production of these metabolites reflects the operation of the entire cycle (See, FIG. 6), which is also consistent with the correlations between ¹³C-labeled citrate, succinate, and Glu in the lung tumor tissues (See, FIG. 5B,C). It is also interesting to note a stronger relationship between ¹³C-labeled Ala and succinate (FIG. 5A) than that between ¹³C-labeled lactate and succinate in lung tumor tissues. This could imply a separate pool of pyruvate derived from Ala for entry into the Krebs cycle (See, FIG. 6B). Although unexpected, the combination of activated glycolysis and Krebs cycle may explain how the excess demand for energy, NADPH reducing equivalents, and biosynthetic precursors (e.g. Asp for nucleotides, citrate for fatty acyl chains, Glu/Gln for proteins) is fulfilled for tumor growth and proliferation.

Metabolomic Data Show Anaplerotic Pathway in Human Lung Tumor is Activated

The display of lung tumor tissues in the enhanced production of the [¹³C₃]-Asp mass isotopomer (Table 4) is intriguing. Given the enhanced expression of PC, this may be explained with the activation of pyruvate carboxylation, i.e. carboxylation of [U-¹³C]-pyruvate to generate [¹³C₃]-oxaloacetate, which is transaminated to produce [¹³C₃]-Asp (FIG. 6B). Alternative paths to Asp synthesis from [U-¹³C]-glucose via glycolysis and the first turn of the Krebs cycle leads to two ¹³C labels in Asp, not the three ¹³C-labeled carbons in Asp that was observed (e.g., Table 4). For the second turn of the Krebs cycle, [¹³C₃]-Asp can be produced, provided that [¹³C₂]-acetyl CoA (derived from [U-¹³C]-pyruvate via PDH) is condensed with [¹³C₂]-OAA from the first turn. However, the % enrichment of acetyl CoA or [U-¹³C]-pyruvate and [¹³C₂]-OAA was low, as evidenced by the ≦15% enrichment in pyruvate surrogate Ala and lactate and <8% enrichment in OAA surrogate Asp. Thus, the probability for the condensation of [¹³C₂]-acetyl CoA in the second turn with [¹³C₂]-OAA from the first turn appears low (<1%). Without wishing to be bound b theory, we suggest that labeled acetyl CoA may always condense with unlabelled OAA, and the labeling pattern of the Krebs cycle intermediates will have the appearance of a single turn, regardless of the actual turn numbers.

Under the low enrichment conditions, the PDH activity produces ¹³C-4,5-αKG (and thus ¹³C-4,5-Glu) and [¹³C₂]-Asp (m0+2) through the Krebs cycle (See, FIG. 6A). In contrast, pyruvate carboxylation leads to the production of ¹³C-2,3-Glu via the Krebs cycle, which is distinct from the PDH pathway (See, FIG. 6B). This distinction in the labeled pattern of Glu provided further metabolic evidence for PC activation in lung tumor tissues. Namely, the ¹³C enrichment of Glu at the C-2 and C-3 positions (FIG. 4B and Table 3) was higher in tumor tissues than its non-cancerous counterpart. Taken together, the ¹³C isotopomer analysis by both NMR and GC-MS revealed the activation of anaplerotic pyruvate carboxylation pathways in non-small cell lung cancer (NSCLC).

Gene Expression Patterns of PC, Glutaminase, and Krebs Cycle Dehydrogenases in Human Lung Tumors

The distinct ¹³C labeling patterns in the Krebs cycle metabolites in tumor tissues described above indicates the possibility of altered gene expression in relevant enzymes. This was examined by real-time PCR analysis as described in the “Gene Expression Analysis” section. Some mitochondrial dehydrogenases (DH) along with the anaplerotic pyruvate carboxylase (PC) and glutaminase for tumor and surrounding non-tumorous tissues are shown in Table 6.

TABLE 6
Changes in gene expression patterns of Krebs cycle and anaplerotic
enzymes in lung tumor versus surrounding non-tumor tissues.

| | Fold Change ^{A} | | | | | | |
|---|---|---|---|---|---|---|---|
| Genes ^{B} | PC | GLS | IDH3 | OGDH | SDH | FH | MDH2 |
| Average ^{C} | 3.36 | 0.52 | 0.67 | 0.58 | 0.93 | 1.01 | 1.53 |
| SD | 1.13 | 0.16 | 0.13 | 0.09 | 0.19 | 0.23 | 0.29 |
| p-value ^{D} | 0.03 | 0.04 | 0.05 | 0.01 | 0.24 | 0.31 | 0.04 |

^{A} Tumor over surrounding non-tumor tissues;
^{B} PC: pyruvate carboxylase; GLS: glutaminase; IDH3: isocitrate dehydrogenase; OGDH: α-ketoglutarate dehydrogenase; SDH: succinate dehydrogenase; FH: fumarate hydratase; MDH2: mitochondrial malate dehydrogenase;
^{C} For patients #6-10;
^{D} Obtained from paired t-test.

Increased expression of the two isoforms of PC gene was evident in patients #6-10 with an average fold change of 3.36±1.13 i.e. higher in tumors (p<0.01). In contrast, the expression of another anaplerotic enzyme gene, glutaminase (GLS) was lower in the tumors relative to the surrounding non-cancerous tissues, with an average fold change of 0.52±0.16. For Krebs cycle DH, there was a decrease of isocitrate DH (IDH) and α-ketoglutarate DH (OGDH) in contrast to a modest activation of malate DH (MDH) expression, while succinate DH (SDH) and fumarate hydratase (FH) showed no statistically significant changes in expression in tumor tissues.

PC Protein Expression Patterns in Human Lung Tumors

The in vivo ¹³C isotopomer profile and gene expression data (see above) indicate increased PC activity in the NSCLC tumors compared with non-tumorous lung tissue. To determine whether PC gene activation leads to an enhanced expression of the enzyme, Western blotting was performed on paired tumor and non-cancerous tissues from the five patients.

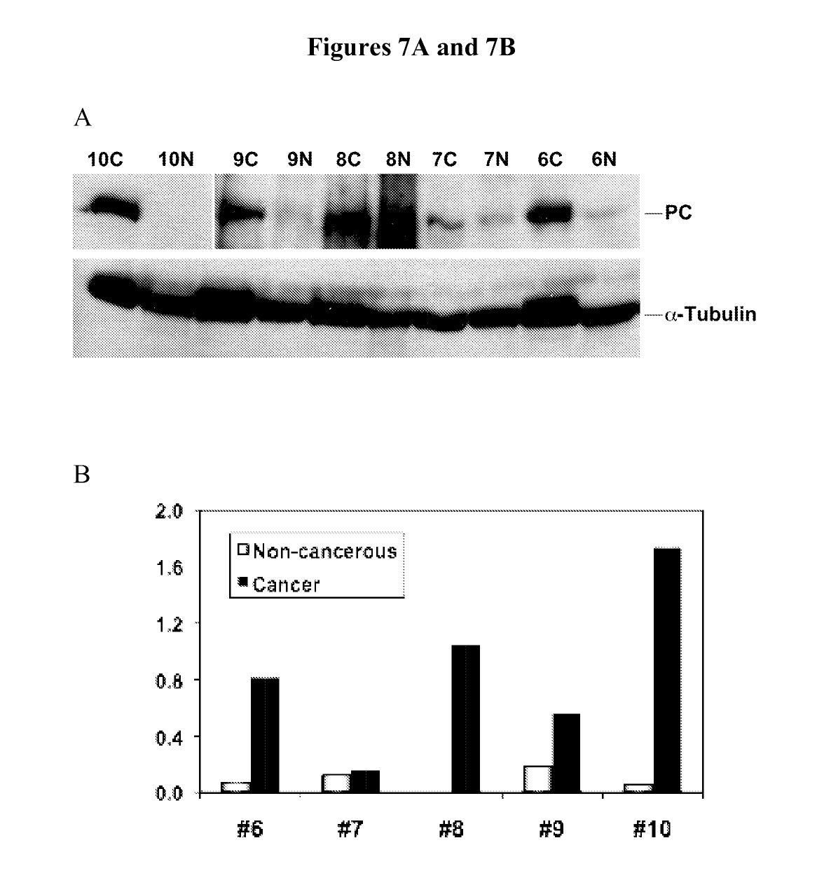

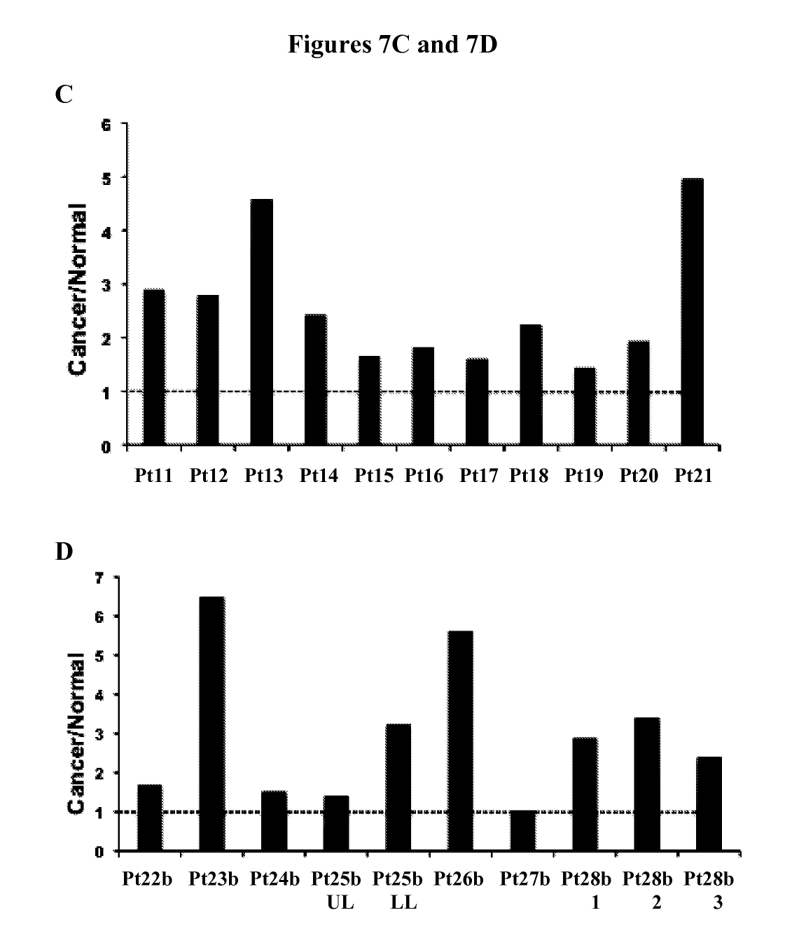

FIG. 7 shows Western blot analysis of PC protein patterns of paired tumor and non-cancerous tissues from patients #6-10. Western blotting (panel A) and image analysis (panels B and C) were performed as described in the Western Blotting of Pyruvate Carboxylase section above. Normalized PC response represented PC image density normalized to α-tubulin image density. The non-cancerous tissue of patient #8 had a high interfering background with no discernable PC band in the blot image, which was not quantified. The cancer tissue of patient #10 had a very intense PC band, which along with the PC band of the non-cancerous tissue was quantified using the blot image with 2 min of film exposure, as was the case for the α-tubulin band of all tissues. PC bands for the rest of tissues were quantified using the same blot but with 17 min of film exposure. N: non-cancerous; C: cancer; ND: not determined. The data shown is representative of two separate blot analyses.

The PC response normalized to that of α-tubulin is shown in FIG. 7B. Lung tumor tissues from patients #6, 9, and 10 exhibited an enrichment of PC protein over the non-cancerous counterpart. Patient #8 had a high interfering background over the PC band region for the non-cancerous tissue, which made it difficult to quantify the PC response. The normalized PC response for patient #7 was comparable between the paired non-cancerous and tumor tissues.

Panel C shows ratios of α-tubulin-normalized Western Blot image analysis of tumor and non-cancerous tissue for patients #11-21 and #22b-28b. The horizontal dotted line represents a ratio of 1. Patient 25b had a lesion on the upper lobe (UL) and a lesion on the lower lobe (LL). Both lesions were at Stage I, but the UL lesion appeared to be in an earlier stage than the LL lesion, because (1) the LL lesion had a higher PET (positron emission tomography) SUV (standardized uptake value), (2) the LL lesion responded to Erlotinib whereas the UL lesion did not, and (3) EGFR analysis was consistent with the UL lesion being in an earlier stage. See also, Fan et al. (2009) Exp. Molec. Pathol. 87, 83-86. Three regions of patient 28b's lung tumor were analyzed; these data suggest that PC expression is consistent for the three regions analyzed.

Gene and Expression Data Show Anaplerotic Pathway in Human Lung Tumor is Activated

The metabolomic data were corroborated by measurements of enhanced gene and protein expression of PC in lung tumors relative to their non-cancerous counterparts.

These effects of PC may be mediated through its ability to replenish the Krebs cycle intermediates, thereby enhancing energy metabolism and fulfilling biosynthetic demands from proliferating cells. Thus, PC activation may play a role in the transformation of lung primary cells into a more highly proliferative state.

B. SCID Mouse Studies

The severe combined immunodeficient (SCID) mouse was used to study the biology of human tumors. The SCID mouse has an impaired ability to make either B or T lymphocytes, or activate some components of the complement system and as such do not reject foreign tissue such as a xenografted human tumor. The mouse tissue fluid volume is relatively small: a 20 g mouse contains ca. 2 ml blood, and a total of ca. 14 ml tissue water. Thus, smaller amounts of isotope enriched precursor metabolites are needed to raise the concentration to a measurable initial value.

We have determined the time course of [U-¹³C]-glucose utilization and metabolic rate in 20 g SCID mice using tail vein injection. Incorporation of ¹³C into metabolites extracted from different tissues have been used to demonstrate the major differences in tissue-dependent metabolism and help ascertain optimal sampling times for different target tissues.

Mouse Handling

The murine tumor therapy protocols were conducted in compliance with all guidelines and were approved by the Institutional Animal Care and Use Committee (IACUC) of the University of Louisville. Fox Chase ICR severe combined immunodeficient (SCID) mice were purchased from Taconic (Hudson, N.Y.) and maintained in a barrier facility at the University of Louisville according to institutional protocols.

Orthotopic Lung Tumor

Human PC14PE6 and A549 cells were grown in RPMI medium as previously described (Fan et al. (2005) Metabolomics 1(4): 1-15), mixed with matrigel (total 100 μl/10⁶ tumor cells) and injected into the left lung. An equal volume of normal saline was injected into the right lung. Mice were allowed to feed ad libitum, and were monitored daily for signs of distress. Mice were sampled at 10 days post injection. Additional naïve SCID mice were used as controls (Onn et al. (2003). Clinical Cancer Research 9(15): 5532-5539).

[U-^{13]}C Glucose Infusion

A 20% solution of glucose in normal saline was sterile-filtered. 100 μL of this solution, or a 1:10 dilution (2%) were injected into the tail vein of a restrained mouse without anesthesia.

Approximately 50 μL samples of blood were taken intraorbitally at timed intervals, chilled and separated into plasma and whole blood by centrifugation at 4° C. at 3,000 g for 5 minutes. Plasma was flash frozen in liquid N₂ for storage prior to workup.

Tissue Harvesting

Mice were sacrificed at different times post glucose injection, and the following organs were dissected sequentially: lung, heart, liver, kidney, brain, spleen, and thigh muscle. Dissected tissue was flash frozen in liquid N₂ within 1 minute of killing the animal.

Plasma Extraction

Twenty to thirty μl of plasma was made to 10% trichloroacetic acid (TCA) and centrifuged at 4° C., at ≧22,000 g for 20 minutes to remove denatured proteins. The polar supernatant was lyophilized to remove TCA before preparation for NMR and GC-MS analysis.

Tissue Extraction

Frozen tissues were ground in liquid N₂ to <10 μm particles in a 6750 Freezer/Mill (Retsch, Inc., Newtown, Pa.) and extracted for soluble and lipidic metabolites as follows. Up to 20 mg of frozen tissue powder in 15 ml polypropylene conical centrifuge tube (Sarstedt, Newton, N.C.) containing 3 mm diameter glass beads was vigorously mixed with 2 ml of cold acetonitrile (mass spectrometry grade, stored at −20° C.) to denature proteins, followed by addition of 1.5 ml nanopure water, and 1 ml HPLC-grade chloroform (Fisher Scientific). The mixture was shaken vigorously until achieving a milky consistency followed by centrifugation at 3,000 g for 20 minutes at 4° C. to separate the polar (top), lipidic (bottom), and tissue debris layers (interface). The polar and lipidic layers were recovered sequentially and the remaining cell debris was extracted again with 0.5 ml chloroform:methanol:butylated hydroxytoluene (BHT) (2:1:1 mM) which was pooled with the lipidic fraction. All three fractions were vacuum-dried in a speedvac device (Vacufuge, Eppendorf, New York, N.Y.) and/or by lyophylization. The dry weight of tissue debris was obtained for normalization of metabolite content. The polar extracts were redissolved in 100% D₂O containing 30 nmol perdeuterated DSS (2,2-dimethyl-2-silapentane-5-sulfonate, Cambridge Isotope Laboratories, Andover, Mass.) as internal chemical shift and concentration reference for NMR measurement.

NMR Spectroscopy

NMR spectra were recorded at 14.1 T on a Varian Inova spectrometer equipped with a 5 mm inverse triple resonance cold probe, at 20° C. 1D NMR spectra were recorded with an acquisition time of 2 s and a recycle time of 5 sec. Concentrations of metabolites and ¹³C incorporation were determined by peak integration of the ¹H NMR spectra referenced to the DSS methyl groups, with correction for differential relaxation, as previously described (See, Lane et al. (2008) Biophysical Tools for Biologists. 84: 541-588; Fan et al. (2008) Progress in NMR Spectroscopy 52: 69-117; Lane et al. (2007) Metabolomics 3: 79-86.). ¹H Spectra were typically processed with zero filling to 131 k points, and apodized with an unshifted Gaussian and a 0.5 Hz line broadening exponential.

¹³C profiling was achieved using 1D ¹H -{¹³C} HSQC spectra recorded with a recycle time of 1.5 s, with ¹³C GARP decoupling during the proton acquisition time of 0.15 s.

TOCSY and HSQC-TOCSY spectra were recorded with a mixing time of 50 ms and a B₁ field strength of 8 kHz with acquisition times of 0.341 s in t₂ and 0.05 s in t₁. The fids were zero filled once in t₂, and linear predicted and zero filled to 4096 points in t₂. The data were apodized using an unshifted Gaussian and a 1 Hz line broadening exponential in both dimensions. Positional ¹³C incorporation into labeled metabolites was quantified as previously described (Lane et al. (2007) Metabolomics 3: 79-86.; Lane et al. (2008) Biophysical Tools for Biologists. 84: 541-588)

Time Course Changes of Plasma ¹³C-Glucose and ¹³C-Lactate

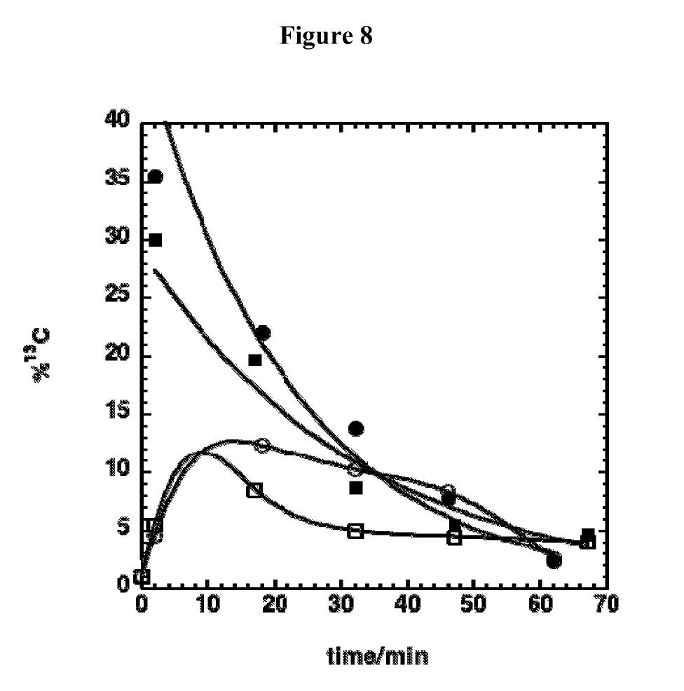

The two SCID mice were injected via the tail vein with 107 mmol [U-¹³C]-glucose. Blood was sequentially taken intraorbitally, and analyzed by NMR as described in the NMR Spectroscopy section. To assess the optimal time for metabolizing the ¹³C₆-glucose in mice, plasma samples were taken at 15-minute intervals and processed for NMR analysis. FIG. 8 shows a representative time course of the % ¹³C enrichment in glucose (solid symbols) and lactate (open symbols) in the plasma of two SCID mice. The initial glucose enrichment ranged from 30 to >50% depending on the size of the mouse, which means that the plasma glucose concentration was roughly doubled immediately after the bolus [U-¹³C]-glucose injection. The % enrichment for glucose decreased rapidly and asymptotically within one hour, presumably via the normal homeostatic mechanism. The apparent half-life of the ¹³C glucose was 16-22 minutes in these mice. The initial rate of ¹³C lactate production was approximately equal to the initial rate of glucose consumption, but then decayed after about 20 minutes.

Once taken up by tissues, labeled glucose is metabolized principally via glycolysis, which generates labeled lactate and to a lesser extent alanine Lactate is then exported into the blood and ultimately to the liver to be reconverted into glucose by gluconeogenesis (Cori Cycle). ¹³C-lactate in plasma therefore represents lactate newly synthesized since the ¹³C glucose bolus, and is an indicator of systemic metabolism. This is consistent with the observed ¹³C enrichment in lactate (FIG. 8), which started low, rapidly reached a maximum, and then depleted over longer periods, reflecting the decrease in the enrichment of the source glucose. The peak enrichment in lactate approached 8-12% at around 15-20 min post injection in this experiment. A larger bolus in which the initial ¹³C glucose enrichment reached 65% was associated with a higher peak ¹³C lactate level (not shown). These data helped determine the optimal duration of tissue harvest for SIRM analysis.

Tissue-Dependent Metabolism: GC-MS Analysis

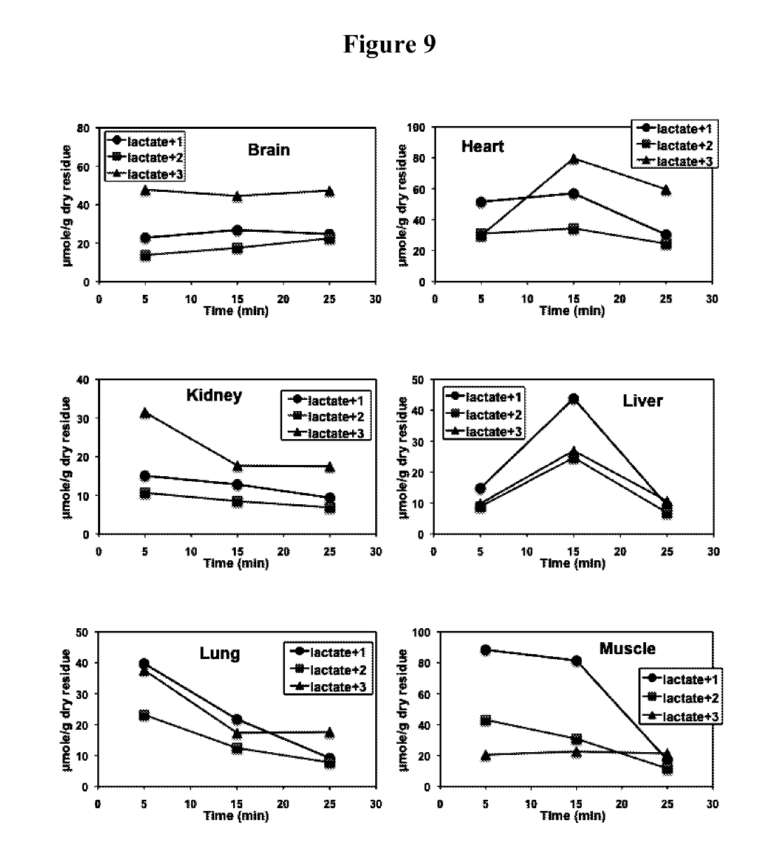

Individual tissues may take up and metabolize glucose at different rates, which will be reflected in the distribution of ¹³C labeled metabolites in various tissues. FIG. 9 shows the GC-MS analysis of ¹³C-lactate isotopologue content of six different tissues dissected from SCID mice 5, 15, and 25 minutes after injection of the ¹³C₆-glucose bolus. The ¹³C₃-lactate (lactate+3) isotopologue showed the highest level in brain, followed by lung and kidney after only 5 minutes of glucose metabolism. Since ¹³C₃-lactate is principally a product of glycolysis, it is expected that the highly glycolytic brain tissue would show the highest initial production of this isotopologue and maintenance of the initial level thereafter. In comparison, the ¹³C-lactate level was lower in lung and kidney while it peaked in heart and liver after 10 minutes of labeled glucose injection (FIG. 9). The time course of ¹³C₃-lactate production in lung and kidney tracked closely with that of the plasma ¹³C₆-glucose level (FIG. 8), which could reflect a high rate of glucose oxidation coupled with a high rate of lactate consumption and/or export in these two organs. The delayed but high production of ¹³C₃-lactate in the heart could reflect its high-energy demand from contraction but preference for drawing energy from 13-oxidation of fatty acids in addition to glucose. Similar to the brain, muscle tissue maintained a constant but lower production of ¹³C₃-lactate from ¹³C₆-glucose, which could be related to a contribution of glycogen metabolism to total lactate production in muscle.

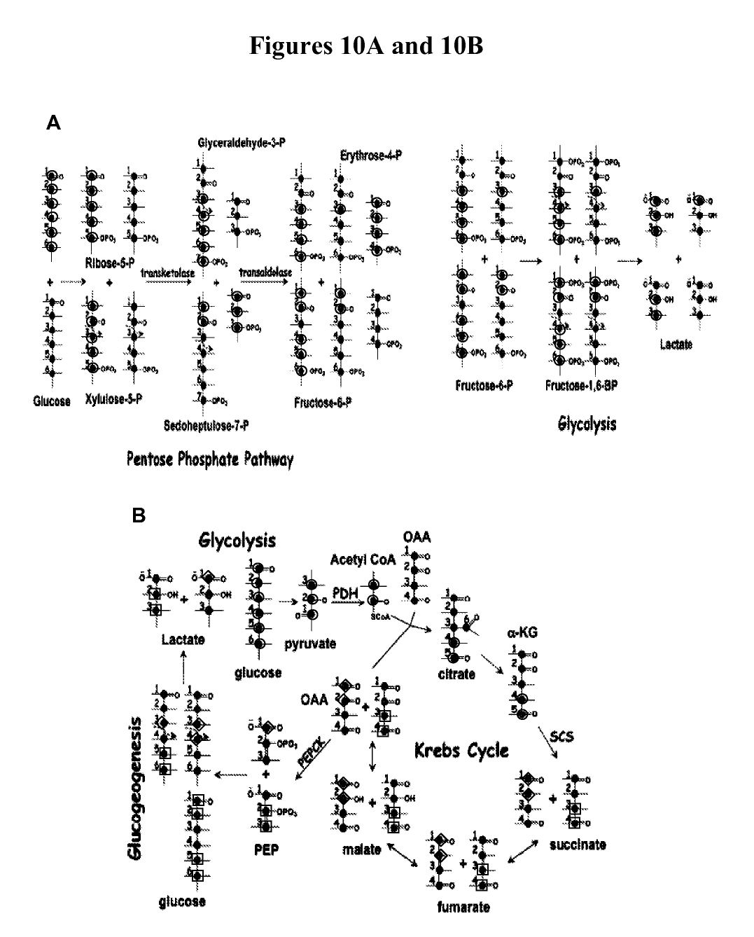

Furthermore, an appreciable amount of singly and doubly ¹³C labeled lactate (lactate+1 and +2 or ¹³C₁- and ¹³C₂-lactate) was observed in all six tissues (FIG. 9). ¹³C₁- and ¹³C₂-lactate built up within 5 minutes of [U-¹³C]-glucose injection and the change in their levels thereafter was small in brain, heart, and kidney. In contrast, ¹³C₁- and ¹³C₂-lactate decreased with time in lung and muscle, while in liver, their levels peaked after 15 minutes of tracer introduction and declined thereafter. Neither ¹³C₁-lactate nor ¹³C₂-lactate can be produced from ¹³C₆-glucose via glycolysis alone. Their production requires metabolic scrambling first through the non-oxidative branch of the pentose phosphate pathway (PPP) (FIG. 10A). Alternatively, in gluconeogenic tissues (i.e. liver and kidney), ¹³C₁- and ¹³C₂-lactate can also be produced from ¹³C₆-glucose via the sequence of glycolysis, Krebs cycle, gluconeogenesis, and glycolysis again (FIG. 10B). Thus, the initial buildup of the scrambled lactate isotopologues in these tissues could reflect the carbon flow through the PPP while the subsequent depletion in lung and muscle could be attributed to a combination of labeled glucose depletion in the plasma and lactate export. The further buildup of scrambled lactate in liver could result from PPP, gluconeogenic activity, and lactate import from the blood.

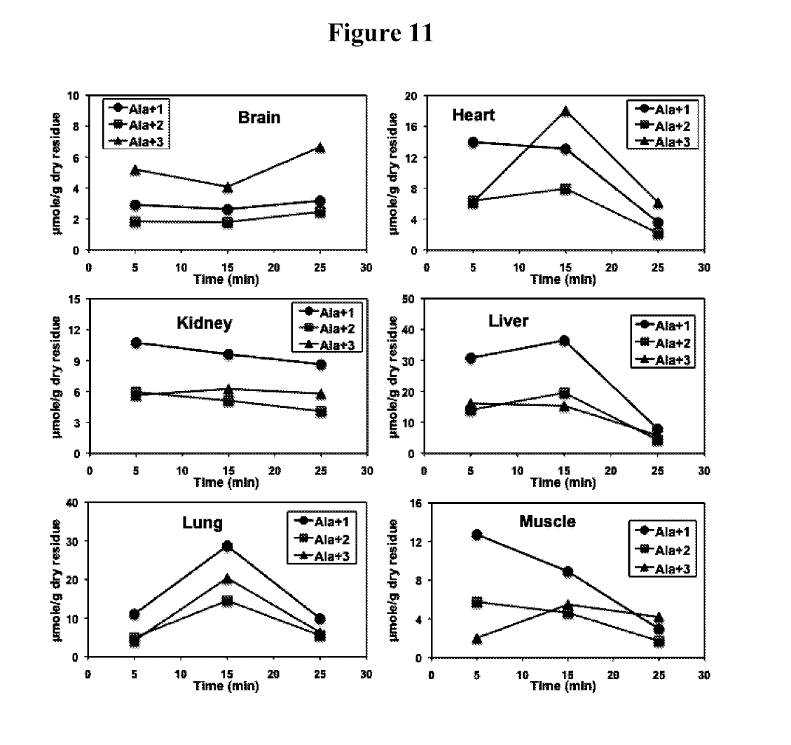

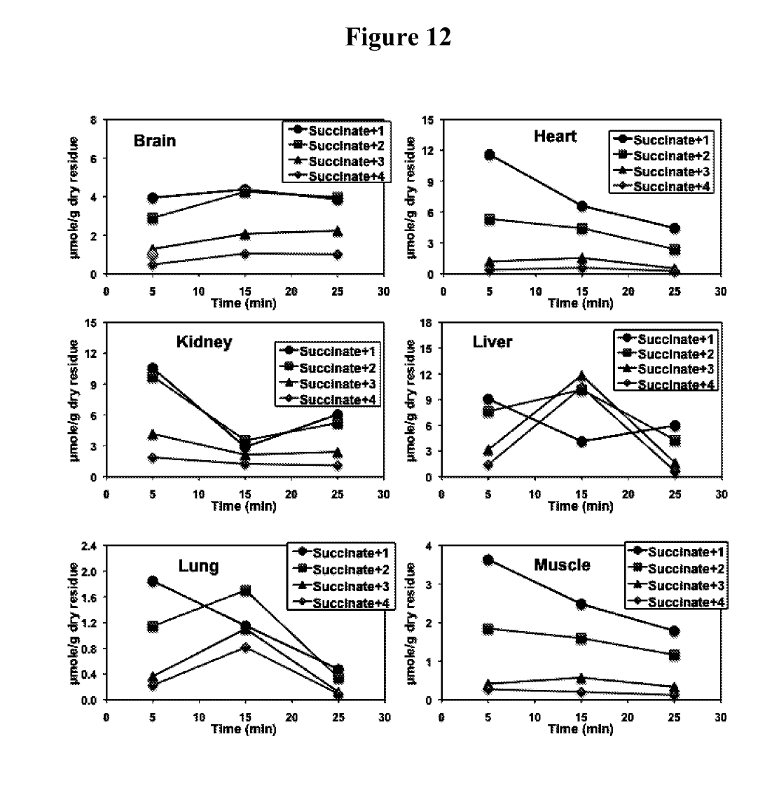

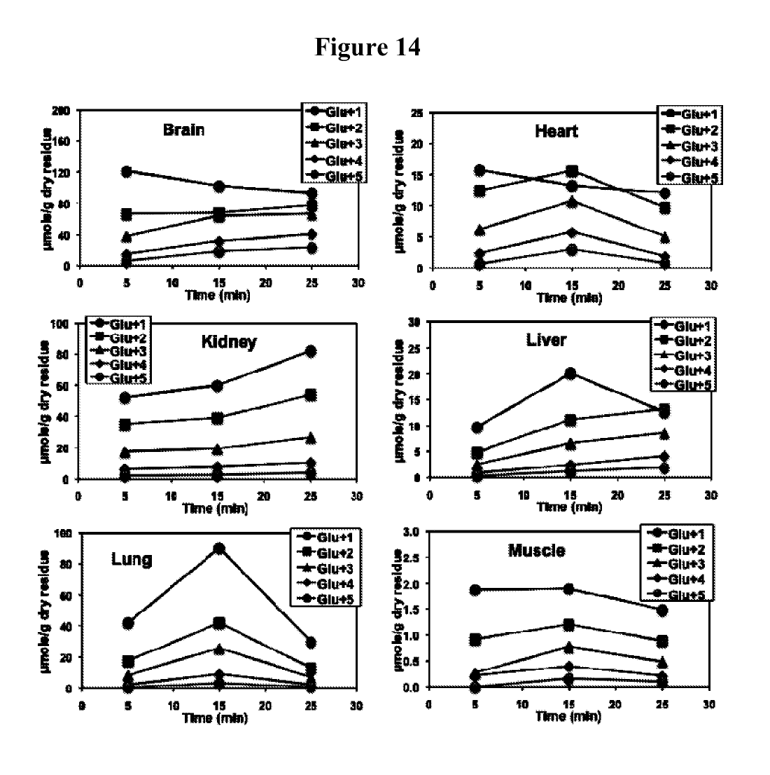

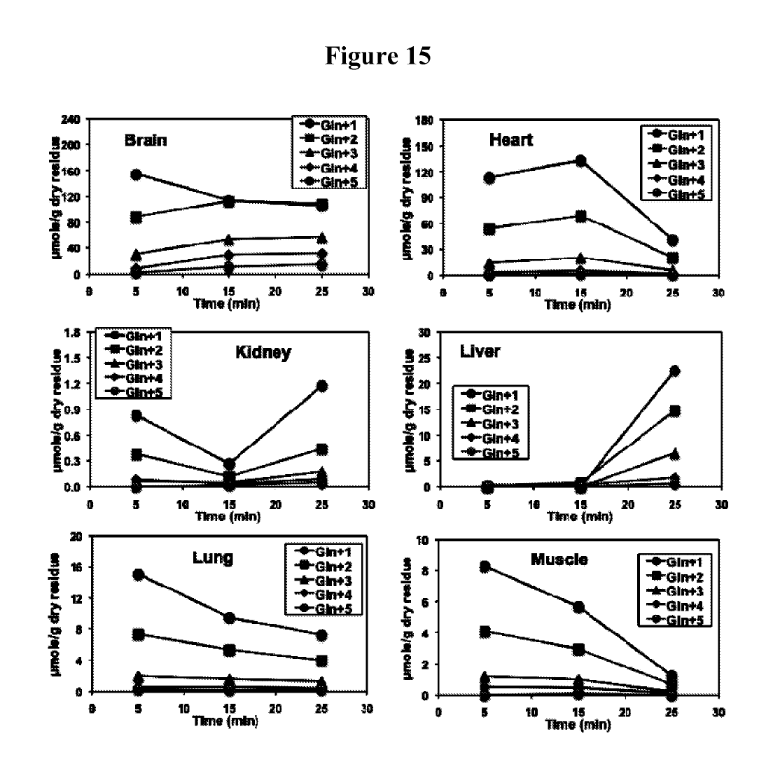

In addition to lactate, ¹³C-labeled isotopologue series for a number of metabolites were observed by GC-MS analysis. FIGS. 11-15 show time course changes measured for Ala, succinate, Asp, Glu, and Gln; similar data were collected, but are not shown for fumarate, malate, citrate, GAB, Ser, Pro, Asn, and Gly. The time course of ¹³C labeling for Ala was similar to that of lactate for all five tissues except for the lung. The level of the three ¹³C isotopologues of Ala in lung peaked at 15 minutes, while that of lactate in the lung declined after 5 minutes of ¹³C₆-glucose injection. Both labeled Ala and lactate share the same precursor, namely labeled pyruvate derived from glycolysis and/or PPP. Yet they differed in their time course behavior in the lung. This implied the presence of two separate pools of pyruvate each for lactate and Ala synthesis. As for lactate, the scrambled ¹³C₂- and ¹³C₂-Ala reflected transformations of ¹³C₆-glucose via PPP and/or gluconeogenesis.

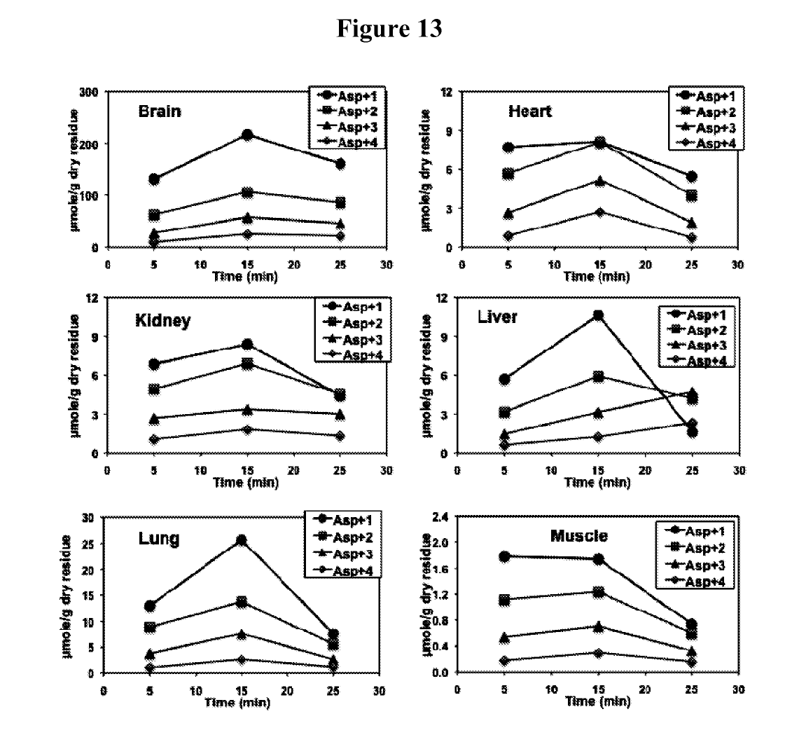

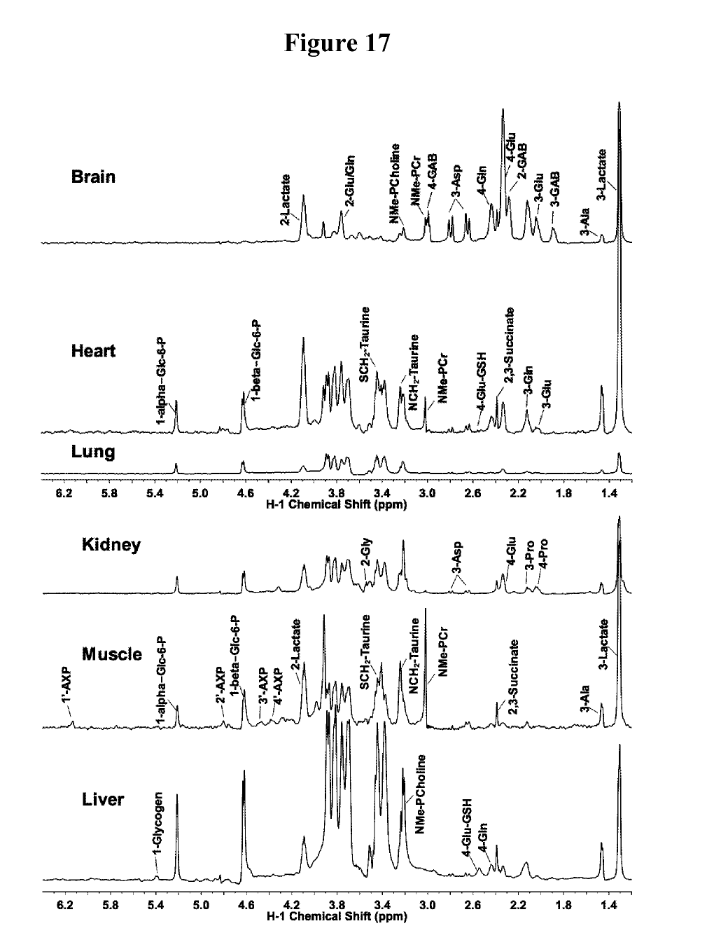

The ¹³C— isotopologue series of succinate, Asp, Glu, and Gln were clearly present and some of which reached high levels, e.g. ¹³C₁-/¹³C₂-/¹³C₃-Asp in brain, ¹³C₁-/¹³C₂-/¹³C₃-/¹³C₄-Glu in brain, kidney, and lung, as well as ¹³C₁-/¹³C₂-Gln in brain and heart. ¹³C₂-succinate, ¹³C₂-Asp, ¹³C₂-Glu, and ¹³C₂-Gln can be derived from ¹³C₆-glucose via glycolysis plus the 1^{st} turn of the Krebs cycle (See, FIG. 10B). The unusual abundance of ¹³C₂-Asp, ¹³C₂-Glu, and ¹³C₂-Gln in the brain suggests a high flux through the Krebs cycle for the production of neurotransmitters, which is consistent with the high buildup of ¹³C₂-GAB (data not shown). There was also an appreciable presence of ¹³C₃-/¹³C₄-Asp, ¹³C₃-/¹³C₄-Glu, and ¹³C₃-/¹³C₄-succinate in all six tissues (FIG. 10B), which could be produced via the 2^{nd} and 3^{rd} turn of the Krebs cycle. However, ¹³C₃-Asp could also be derived from the carboxylation of ¹³C₃-pyruvate (PC) via the anaplerotic pyruvate carboxylase activity. PC coupled with the 1^{st} turn of the Krebs cycle would lead to the production of ¹³C₃-citrate while ¹³C₄- and ¹³C₅-citrate would be the expected products from the 2^{nd} and 3^{rd} turn of the Krebs cycle activity, respectively. ¹³C₃-citrate was present at a higher level than those of ¹³C₄- and ¹³C₅-citrate in all six tissues (data not shown), which suggests that PC contributed significantly to the production of ¹³C₃-Asp in these tissues. The high PC activity in the brain is supported by the high level of ¹³C₃-Asp (FIG. 13) and abundance of ¹³C-2-Glu and ¹³C-3-Glu (FIG. 17), which can be derived from ¹³C₆-glucose via glycolysis, PC, and 1^{st} turn of the Krebs cycle.

Tissue-Dependent Metabolism: NMR Analysis

The same extracts from FIG. 9 were analyzed by NMR to complement the GC-MS analysis. The ¹³C enrichment into glucose/glucose-6-phosphate and lactate was determined by 1D ¹H NMR. The enrichments at 15 and 30 minutes post infusion were substantial in all tissues, and peak lactate enrichments differed according to the metabolic activity or glucose uptake rates of the different tissues (Table 7).

TABLE 7
Isotopic enrichment in tissue lactate and glucose

| Tissue | Time/min | % ¹³C Lac | % ¹³C Glc |
|---|---|---|---|
| Heart | 30 | 33 | 28 |
| | 15 | 25 (Ala = 16) | 19 |
| Liver | 30 | 13 | 6 |
| | 15 | 11 | 4.5 |
| Thigh muscle | 30 | 11 | 11 |
| | 15 | 17 | 15 |
| Kidney | 30 | 17 | ND |
| | 15 | 19 | 22 |
| Lung N | 30 | 17 | 14 |
| | 15 | 20 | 22 |

Lac measured at C3, G1c measured at C1α by NMR

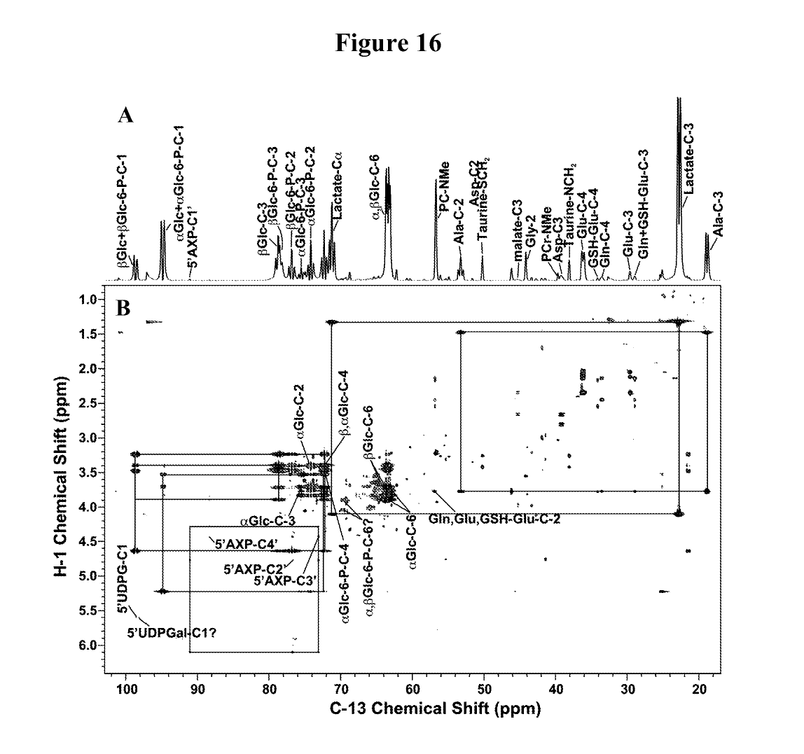

More extensive ¹³C metabolite labeling was determined by two-dimensional NMR experiments. FIG. 16 shows a high-resolution 2-D ¹H-¹³C HSQC-TOCSY (heteronuclear single quantum coherence-total correlation spectroscopy) spectrum (panel B) of a lung extract obtained from a SCID mouse 15 minutes after injecting ¹³C₆-glucose. ¹³C₆-glucose was infused into SCID mouse via tail vein and lung tissue was dissected, pulverized, and extracted as described above. The 2-D spectrum as contour plot (panel B) was acquired at 14.1 T, processed with linear prediction in the ¹³C dimension and zero-filling to 4k×2k real digital points. Also displayed is the 1-D projection spectrum along the ¹³C dimension (panel A). Sufficient time-resolved data were acquired in the ¹³C dimension to delineate the ¹³C splitting pattern of each resonance. The ¹³C coupling patterns of labeled resonances can be visualized in the 1-D projection spectrum onto the ¹³C dimension (panel A). The HSQC-TOCSY data not only confirmed the identity of ¹³C-labeled metabolites by their characteristic ¹H-¹³C and ¹H-¹H covalent linkages but also enabled determination of the labeled carbon position, i.e. positional isotopomers. For example, the lactate was confirmed by the covalent linkages represented by cross-peaks from H-3 to C-3, H-2 to C-2, and H-3 to H-2 of lactate (FIG. 16B). The ¹³C-coupling pattern of C-3 and C-2 of lactate was respectively doublet and triplet (FIG. 16A), indicating that lactate was predominantly labeled at all three carbon positions. This is consistent with the abundance of ¹³C₃-lactate by GC-MS analysis of the same extract (FIG. 9). Similarly, the doublet pattern of C-1 and C-6 and triplet pattern of C-2, C-3, and C-4 of glucose-6-phosphate (Glc-6-P) indicate the presence of ¹³C₆-Glc-6-P in the extract, which derives from ¹³C₆-glucose via hexose kinase activity. This information and the labeled patterns of glutathione, adenine nucleotides, and UDP-sugars was not obtainable by GC-MS. Moreover, the N-methyl carbons of phosphocholine and C-2 of Gly were singlets, which suggests that they may be largely contributed from the natural abundance ¹³C, and therefore not derived from ¹³C₆-glucose.

Once the labeled metabolites were assigned by 2-D HSQC methods, the 1-D HSQC analysis of ¹H directly attached to ¹³C provided a semi-quantitative estimate of the abundance of various ¹³C-labeled metabolites in the six tissues (FIG. 17). SCID mice were infused with ¹³C₆-glucose for 15 minutes before tissue dissection, extraction, and analysis by NMR as described in above. Different tissues appear to exhibit distinct patterns of glucose metabolism, as reflected by the different 1-D HSQC profiles. Brain tissue built up a significant level of ¹³C-3-Asp, ¹³C-2, 3, or 4-Glu/Gln and ¹³C-2, 3, or 4-γ-aminobutyrate (GAB), which is consistent with its high Krebs cycle activity and specialized production of neurotransmitter Glu, Glu, and GAB. There was no detectable ¹³C-labeled free glucose in the brain tissue, which could be related to its high glycolytic activity (FIG. 9) and high requirement for glucose in energy production. Heart tissue was also high in labeled Glu, Gln, and succinate, which reflects a high Krebs cycle activity. It also had the highest labeled lactate level among the six tissues and an appreciable level of labeled Glc-6-P, which suggests a high glucose uptake and glycolytic rates. Liver tissue had the highest Glc-6-P but a moderate level of labeled lactate, which could be a result of fast glucose uptake and gluconeogenesis from lactate. Liver was the only tissue where labeled glycogen was observed, which could be attributed to its high capacity of glycogen synthesis. Muscle tissue had the second highest labeled lactate level, which is consistent with a high rate of glycolysis. In addition, it was the only tissue where the ribose moiety of adenine nucleotides (AXP) was labeled, which suggests that muscle possesses high PPP activity. This is consistent with the high levels of scrambled labeled lactate in the muscle tissue (FIG. 9). Lung and kidney tissues had the lowest overall labeled metabolite content, which suggests a lower rate of glucose metabolism.

Metabolism in Normal Versus Cancerous Lung Tissues

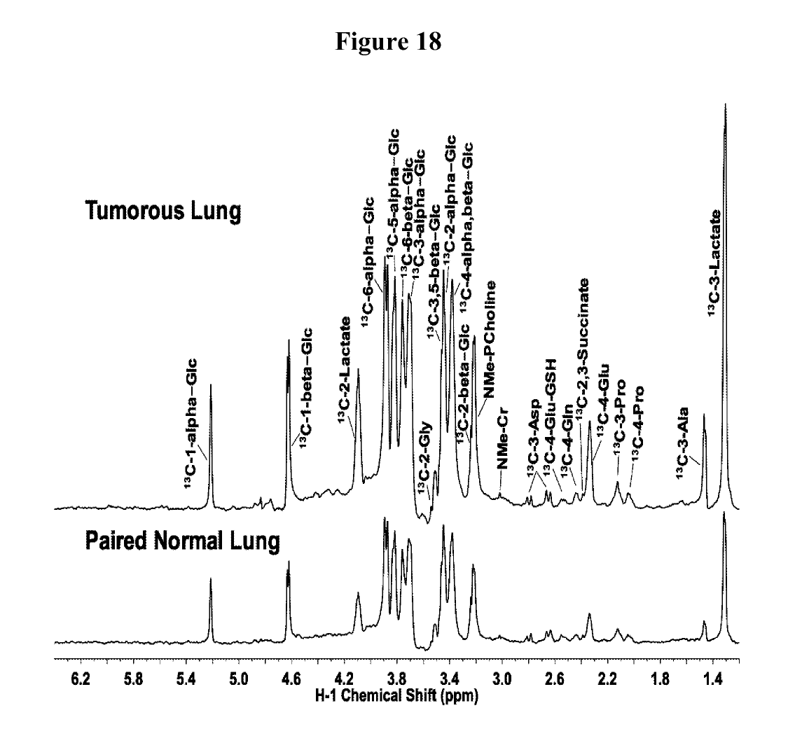

¹³C₆-glucose was used as a tracer to track changes in metabolic pathways induced by lung tumor development in SCID mice. An SCID mouse was injected with 1 million PC14PE6 lung cancer cells in matrigel in one of the lung lobes while the other lung lobe received saline only. After lung tumor establishment, the animal received ¹³C₆-glucose 15 minutes prior to tissue dissection, extraction, and NMR analysis as described above. FIG. 18 illustrates the 1-D HSQC comparison of a tumorous lung with its paired normal lung dissected from the same mouse 15 minutes after the [U-¹³C]-glucose infusion. The ¹³C abundance of various positional isotopomers of metabolite (as reflected by the resonance intensity of their protons attached to ¹³C) were higher in tumorous lung than its normal counterpart. These included ¹³C-2 and 3-lactate, ¹³C-3-Ala, ¹³C-3 and 4-Pro, ¹³C-4-Glu, ¹³C-4-Gln, ¹³C-4-glutamyl moiety of GSH (¹³C-4-Glu-GSH), ¹³C-2,3-succinate, ¹³C-2-Gly, ¹³C-1 to 6-glucose, and N-methyl-phosphocholine (NMe-PCholine). The NMe-PCholine signal was attributed to natural abundance of ¹³C since the choline moiety is not expected to be synthesized from ¹³C₆-glucose

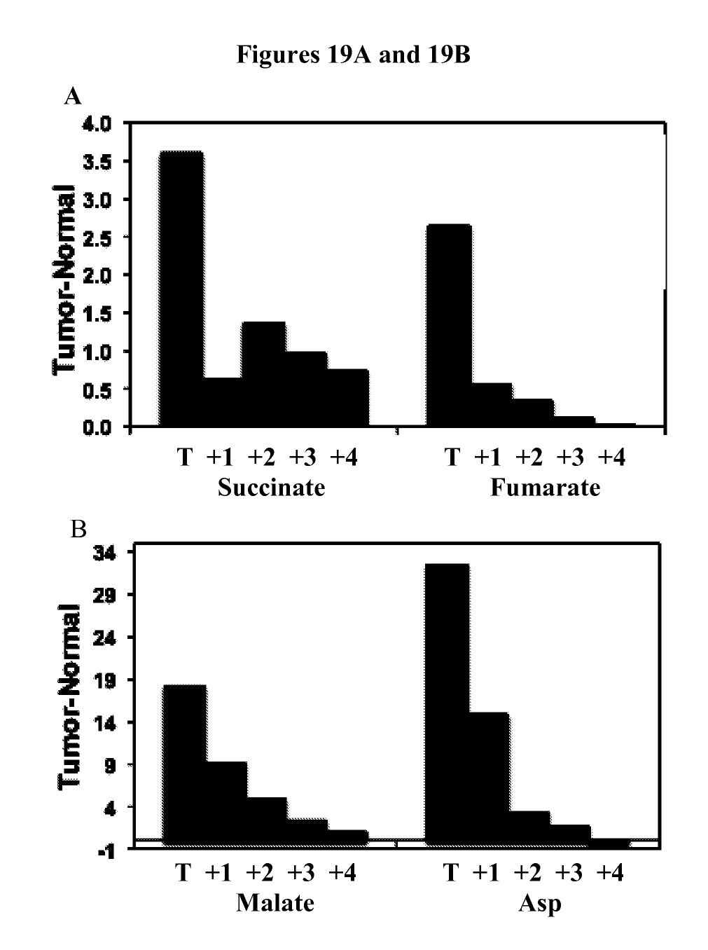

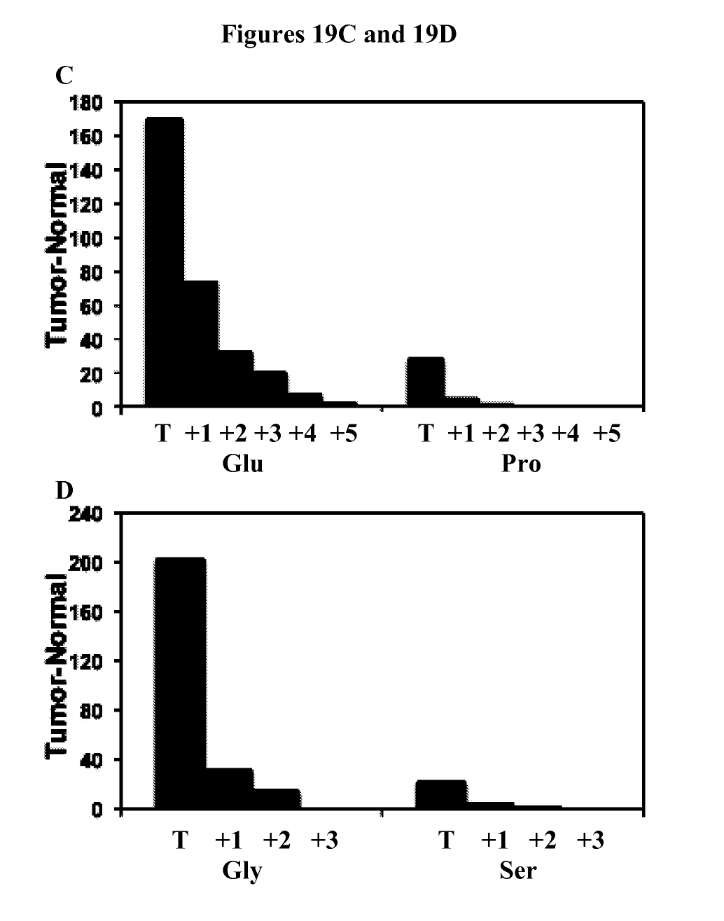

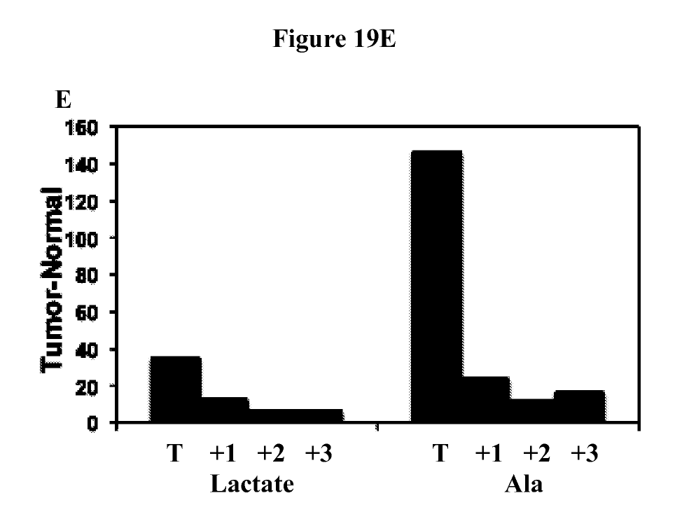

A separate set of tumorous and normal lung tissue extracts were quantified by GC-MS, as shown in FIG. 19. The tumorous and normal lung tissues were obtained from two SCID mice and were similarly prepared and treated as those in FIG. 18. Their polar extracts were analyzed by GC-MS as described above. The GC-MS quantification supported the NMR analysis in terms of the greater production of ¹³C labeled lactate, Ala, succinate, Asp, Glu, Pro, and Gly in the tumorous than in normal lung tissues. In addition, the GC-MS analysis provided evidence for the higher buildup of ¹³C-labeled malate, fumarate, and Ser in the tumorous relative to the normal lung tissues. These metabolites were obscured in the 1-D HSQC spectra in FIG. 18. Moreover, as described above, increased carbon flow through specific pathways in the tumorous lung could be deduced based on the combined ¹³C mass isotopologue series of metabolites acquired from GC-MS and the ¹³C positional isotopomer information obtained from the NMR analysis. For example, the increased production of ¹³C₃-lactate and -Ala is consistent with enhanced glycolysis while the larger buildup of ¹³C₁ and ¹³C₂-lactate and -Ala suggests more active PPP in the tumorous lung. Enhanced Krebs cycle activity appeared evident from the greater accumulation of ¹³C₂-Asp, -succinate, -fumarate, and -Glu in the tumorous lung. Activation of PC in the tumorous lung tissue could be inferred from the increased buildup of ¹³C₃-Asp (FIG. 19B) and the presence of ¹³C₃-citrate isotopologue (data not shown), which is unique product of PC. The higher synthesis of ¹³C₂-Gly in the tumorous lung suggests a more active one-carbon metabolism associated with tumor development.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as a basis for the claims and as a representative basis for teaching one skilled in the art to employ the present invention in any appropriate manner.

What is claimed is:

1. A method for detecting the presence of cancer in an animal that was administered an administered labeled molecule, comprising:
(A) performing (i), (ii), or both:
(i) obtaining a first NMR spectrum of a first non-cancer cell extract, and obtaining a second NMR spectrum of a first cancer cell extract;
(ii) obtaining a first MS spectrum of a second non-cancer cell extract, and obtaining a second MS spectrum of a second cancer cell extract;
(B) determining a first amount of at least one resultant labeled molecule from the first NMR spectrum, from the first MS spectrum, or from both;
(C) determining a second amount of at least one resultant labeled molecule from the second NMR spectrum, from the second MS spectrum, or from both; and
(D) detecting the presence of cancer by comparing the first amount of at least one resultant labeled molecule with the second amount of at least one resultant labeled molecule;
Where:
the first non-cancer cell extract was obtained from a first set of non-cancer cells removed from a tissue of the animal,
the first cancer cell extract was obtained from a first set of cancer cells removed from the tissue of the animal,
the second non-cancer cell extract was obtained from a second set of non-cancer cells removed from the tissue of the animal, and
the second cancer cell extract was obtained from a second set of cancer cells removed from the tissue of the animal.

2. The method of claim 1, wherein the administered labeled molecule was administered to the animal by one or more routes selected from the group consisting of an oral route, a parenteral route, a cutaneous route, a nasal route, a rectal route, a vaginal route, and an ocular route.

3. The method of claim 1, wherein the administered labeled molecule is selected from the group consisting of a ¹³C isotopomer of glucose, a ¹³C isotopomer of pyruvate, a ¹³C isotopomer of Ala, an ¹⁵N isotopomer of Ala, a ¹³C isotopomer of acetate, a ¹³C isotopomer of glutamine, and an ¹⁵N isotopomer of glutamine.

4. The method of claim 1, wherein the administered labeled molecule is selected from the group consisting of [U-¹³C]-glucose, ¹³C₁-glucose, ¹³C₂-glucose, ¹³C₃-glucose, ¹³C₄-glucose, ¹³C₅-glucose, [U-¹³C]-pyruvate, ¹³C₁-pyruvate, ¹³C₂-pyruvate, [U-¹³C]-Ala, ¹³C₁-Ala, and ¹³C₂-Ala.

5. The method of claim 1, wherein the administered labeled molecule is selected from the group consisting of [U-¹³C]-glucose, [U-¹³C]-pyruvate, [U-¹³C]-Ala, and [U-¹³C]-glutamine.

6. The method of claim 1, wherein the tissue is selected from the group consisting of connective tissue, muscle tissue, nervous tissue, and epithelial tissue.

7. The method of claim 1, wherein the tissue is at least part of an organ selected from the group consisting of heart, blood, blood vessel, salivary gland, esophagus, stomach, liver, gallbladder, pancreas, large intestine, small intestine, appendix, rectum, anus, colon, endocrine gland, kidney, ureter, bladder, urethra, skin, hair, nail, lymph, lymph node, lymph vessel, leukocyte, erythrocyte, tonsil, adenoid, thymus, spleen, muscle, skeletal muscle, smooth muscle, breast, brain, spinal cord, peripheral nerve, nerve, sex organ, pharynx, larynx, trachea, bronchi, alveoli, lung, diaphragm, bone, cartilage, ligament, and tendon.

8. The method of claim 1, wherein the tissue is part of an organ system selected from the group consisting of circulatory system, digestive system, endocrine system, excretory system, integumentary system, lymphatic system, muscular system, nervous system, reproductive system, respiratory system, and skeletal system.

9. The method of claim 1, wherein the tissue is from one breast of the animal, both breasts of the animal, one lung of the animal, or both lungs of the animal.

10. The method of claim 1, wherein the tissue from which the first set of non-cancer cells was removed is contralateral to the tissue from which the first set of cancer cells was removed.

11. The method of claim 1, wherein the tissue from which the second set of non-cancer cells was removed is contralateral to the tissue from which the second set of cancer cells was removed.

12. The method of claim 1, wherein the first non-cancer cell extract is obtained by one or more extractions with one or more solutions comprising acetonitrile, water, chloroform, methanol, butylated hydroxytoluene, trichloroacetic acid, or combinations thereof.

13. The method of claim 1, wherein the second non-cancer cell extract is obtained by one or more extractions with one or more solutions comprising acetonitrile, water, chloroform, methanol, butylated hydroxytoluene, trichloroacetic acid, or combinations thereof.

14. The method of claim 1, wherein the first non-cancer cell extract is the same as the second non-cancer cell extract.

15. The method of claim 1, wherein the first cancer cell extract is obtained by one or more extractions with one or more solutions comprising acetonitrile, water, chloroform, methanol, butylated hydroxytoluene, trichloroacetic acid, or combinations thereof.

16. The method of claim 1, wherein the second cancer cell extract is obtained by one or more extractions with one or more solutions comprising acetonitrile, water, chloroform, methanol, butylated hydroxytoluene, trichloroacetic acid, or combinations thereof.

17. The method of claim 1, wherein the first cancer cell extract is the same as the first cancer cell extract.

18. The method of claim 1, wherein the animal is selected from the group consisting of human, dog, cat, horse, cow, pig, sheep, chicken, turkey, mouse, and rat.

19. The method of claim 1, wherein the animal is mammalian.

20. The method of claim 1, wherein the cancer is selected from the group consisting of carcinomas, sarcomas, hematologic cancers, neurological malignancies, basal cell carcinoma, thyroid cancer, neuroblastoma, ovarian cancer, melanoma, renal cell carcinoma, hepatocellular carcinoma, breast cancer, colon cancer, lung cancer, pancreatic cancer, brain cancer, prostate cancer, chronic lymphocytic leukemia, acute lymphoblastic leukemia, rhabdomyosarcoma, Glioblastoma multiforme, meningioma, bladder cancer, gastric cancer, Glioma, oral cancer, nasopharyngeal carcinoma, kidney cancer, rectal cancer, lymph node cancer, bone marrow cancer, stomach cancer, uterine cancer, leukemia, basal cell carcinoma, and cancers related to epithelial cells.

21. The method of claim 1, wherein the cancer is selected from a cancer that can alter the regulation or activity of pyruvate carboxylase.

22. The method of claim 1, wherein the cancer is a cancerous tumor.

23. The method of claim 1, wherein the at least one resultant labeled molecule comprises an isotopomer selected from the group consisting of lactate, alanine (Ala), arginine (Arg), serine (Ser), proline (Pro), asparagine (Asn), Glycine (Gly), glutamate (Glu), oxidized glutathione (GSSG), Glu-GSSH, Glu-GSH, glutamine (Gln), γ-aminobutyrate (GAB), succinate, citrate, isocitrate, fumarate, malate, aspartate (Asp), creatine (Cr), oxaloacetate (OAA), α-ketoglutarate (αKG), phosphocholine (P-choline), N-methyl-phosphocholine, taurine, glycogen, phenylalanine (Phe), tyrosine (Tyr), myo-inositol, α- and β-glucose, NAD⁺, cytosine nucleotides (CXP), uracil nucleotides (UXP), guanine nucleotides (GXP), and adenine nucleotides (AXP).

24. The method of claim 1, wherein the at least one resultant labeled molecule comprises a molecule selected from the group consisting of [U-¹³C]-lactate, [U-¹³C]-Ala, ¹³C-3-Glu, ¹³C-3-Gln, ¹³C-3-glutamyl residue of oxidized glutathione (Glu-GSSG), ¹³C-3-lactate, ¹³C-2-Glu-GSSG, ¹³C-2-Glu, ¹³C-2-Asp, ¹³C-3-Asp, ¹³C-2,3-succinate, ¹³C-2,4-citrate, ¹³C-1′-ribose-5′AXP, ¹³C-1-α-glucose, ¹³C-1-β-glucose, ¹³C-3-Ala, ¹³C-2,3-lactate, ¹³C-3-Gln+GSSG, ¹³C-2 to 4-Glu, ¹³C-4-Gln, ¹³C-4-GSSG, ¹³C-2,4-citrate, ¹³C-2,3-Asp, ¹³C-1′,4′,5′-5′-AXP, and ¹³C-1′,4′-5′-UXP.

25. The method of claim 1, wherein the at least one resultant labeled molecule comprises isotopologues.

26. The method of claim 1, wherein the at least one resultant labeled molecule comprises a collection of molecules selected from the group consisting of ¹³C₂-lactate, ¹³C₃-lactate, ¹³C₂-Ala, ¹³C₃-Ala, ¹³C₂-succinate, ¹³C₃-succinate, ¹³C₄-succinate, ¹³C₂-Asp, ¹³C₃-Asp, ¹³C₄-Asp, ¹³C₂-Glu, ¹³C₃-Glu, ¹³C₄-Glu, ¹³C₅-Glu, ¹³C₂-Gln, ¹³C₃-Gln, ¹³C₄-Gln, ¹³C₅-Gln, ¹³C₂-fumarate, ¹³C₃-fumarate, ¹³C₄-fumarate, ¹³C₂-malate, ¹³C₃-malate, ¹³C₄-malate, ¹³C₂—Pro, ¹³C₃—Pro, ¹³C₄—Pro, ¹³C₅-Pro, ¹³C₂-Gly, ¹³C₃-Gly, ¹³C₂-Ser, ¹³C₃-Ser, ¹³C₂-pyruvate, ¹³C₃-pyruvate, ¹³C₂-citrate, ¹³C₃-citrate, ¹³C₂-isocitrate, and ¹³C₃-isocitrate.

27. The method of claim 1, wherein the first NMR spectrum is selected from the group consisting of 1-D ¹H, 1-D ¹³C, 1-D ¹⁵N, TOCSY, COSY, NOESY, EXSY, and heteronuclear correlation scalar coupling experiments.

28. The method of claim 1, wherein the first NMR spectrum is selected from the group consisting of HSQC, ¹H-¹³C₂-D HSQC, ¹H1-D HSQC, SE-HSQC, CT-HSQC, HSQC-TOCSY, ¹H-¹³C₂-D HSQC-TOCSY, TROSY, HETCOR, INADEQUATE, HMQC, and HMBC.

29. The method of claim 1, wherein the first NMR spectrum is selected from the group consisting of 1-D spectra, 2-D spectra, 3-D spectra, and 4-D spectra.

30. The method of claim 1, wherein the first MS spectrum is obtained using a mass spectrometry system that is a GC/MS or an LC/MS.

31. The method of claim 1, wherein the second NMR spectrum is selected from the group consisting of 1-D ¹H, 1-D ¹³C, 1-D ¹⁵N, TOCSY, COSY, NOESY, EXSY, and heteronuclear correlation scalar coupling experiments.

32. The method of claim 1, wherein the second NMR spectrum is selected from the group consisting of HSQC, ¹H-¹³C₂-D HSQC, ¹H1-D HSQC, SE-HSQC, CT-HSQC, HSQC-TOCSY, ¹H-¹³C₂-D HSQC-TOCSY, TROSY, HETCOR, INADEQUATE, HMQC, and HMBC.

33. The method of claim 1, wherein the second NMR spectrum is selected from the group consisting of 1-D spectra, 2-D spectra, 3-D spectra, and 4-D spectra.

34. The method of claim 1, wherein the second MS is obtained using a mass spectrometry system that is an GC/MS or a LC/MS.

35. The method of claim 1, wherein both (i) and (ii) are performed.

36. The method of claim 1, wherein the method further comprises determining the protein expression of at least one protein in the first set of cancer cells, in the first set of non-cancer cells, in the second set of cancer cells, in the second set of non-cancer cells, or combinations thereof.

37. The method of claim 36, wherein the determining of the protein expression is accomplished using Western blotting or measurement of enzyme activity.

38. The method of claim 1, wherein the method further comprises determining the gene expression of at least one protein in the first set of cancer cells, in the first set of non-cancer cells, in the second set of cancer cells, in the second set of non-cancer cells, or combinations thereof.

39. The method of claim 38, wherein the determining of gene expression comprises using real-time PCR.


SEQUENCE LISTING (16 sequences)

SEQ ID NO: 1 - DNA, 21 bp, Artificial Sequence
  primer sequence
tctggtcctcggtcaggtctg

SEQ ID NO: 2 - DNA, 23 bp, Artificial Sequence
  primer sequence
gacagtgacagcaacatggttcc

SEQ ID NO: 3 - DNA, 24 bp, Artificial Sequence
  primer sequence
gcacagacatggttggtatattag

SEQ ID NO: 4 - DNA, 21 bp, Artificial Sequence
  primer sequence
agaagtcatacatgccacagg

SEQ ID NO: 5 - DNA, 22 bp, Artificial Sequence
  primer sequence
caactgccccttctcctatccc

SEQ ID NO: 6 - DNA, 20 bp, Artificial Sequence
  primer sequence
agcccaagcctaagcccaag

SEQ ID NO: 7 - DNA, 21 bp, Artificial Sequence
  primer sequence
cggaggtggtcaaggctaaag

SEQ ID NO: 8 - DNA, 20 bp, Artificial Sequence
  primer sequence
cagcggtgtggagaagtagg

SEQ ID NO: 9 - DNA, 20 bp, Artificial Sequence
  primer sequence
gtgagaatggcgtggactac

SEQ ID NO: 10 - DNA, 21 bp, Artificial Sequence
  primer sequence
cgattgatcctgcggtgatac

SEQ ID NO: 11 - DNA, 20 bp, Artificial Sequence
  primer sequence
gcgtgtttgactacagtgag

SEQ ID NO: 12 - DNA, 20 bp, Artificial Sequence
  primer sequence
tcttgacctccttgaacttg

SEQ ID NO: 13 - DNA, 21 bp, Artificial Sequence
  primer sequence
catcgcataagagcaaagaac

SEQ ID NO: 14 - DNA, 20 bp, Artificial Sequence
  primer sequence
ccttccgtaatgagacaacc

SEQ ID NO: 15 - DNA, 21 bp, Artificial Sequence
  primer sequence
atcagataccgtcgtagttcc

SEQ ID NO: 16 - DNA, 23 bp, Artificial Sequence
  primer sequence
ccgtcaattcctttaagtttcag